SEQUENCE LISTING (39 sequences)

SEQ ID NO: 1 - protein, 776 aa, Caenorhabditis elegans
  Systemic RNA Interference Defective family member (sid-1)
MIRVYLIILMHLVIGLTQNNSTTPSPIITSSNSSVLVFEISSKMKMIEKKLEANTVHVLRLELDQSFILDLTKVAAEIVDSSKYSKEDGVILEVTVSNGRDSFLLKLPTVYPNLKLYTDGKLLNPLVEQDFGAHRKRHRIGDPHFHQNLIVTVQSRLNADIDYRLHVTHLDRAQYDFLKFKTGQTTKTLSNQKLTFVKPIGFFLNCSEQNISQFHVTLYSEDDICANLITVPANESIYDRSVISDKTHNRRVLSFTKRADIFFTETEISMFKSFRIFVFIAPDDSGCSTNTSRKSFNEKKKISFEFKKLENQSYAVPTALMMIFLTTPCLLFLPIVINIIKNSRKLAPSQSNLISFSPVPSEQRDMDLSHDEQQNTSSELENNGEIPAAENQIVEEITAENQETSVEEGNREIQVKIPLKQDSLSLHGQMLQYPVAIILPVLMHTAIEFHKWTTSTMANRDEMCFHNHACARPLGELRAWNNIITNIGYTLYGAIFIVLSICRRGRHEYSHVFGTYECTLLDVTIGVFMVLQSIASATYHICPSDVAFQFDTPCIQVICGLLMVRQWFVRHESPSPAYTNILLVGVVSLNFLISAFSKTSYVRFIIAVIHVIVVGSICLAKERSLGSEKLKTRFFIMAFSMGNFAAIVMYLTLSAFHLNQIATYCFIINCIMYLMYYGCMKVLHSERITSKAKLCGALSLLAWAVAGFFFFQDDTDWTRSAAASRALNKPCLLLGFFGSHDLWHIFGALAGLFTFIFVSFVDDDLINTRKTSINIF

SEQ ID NO: 2 - protein, 508 aa, Schistosoma japonicum
  sjsid-c, SID-1 related C
MESRKKNRYFTGLNISTTSVDTNTTTGIILDNKQLPTSSAYSTMIGQETSHTPKRSVDLHPSNATNNANNSNDKHRNDKSDNVSSVSLTDEQTDINSKFIPMYKMNVLLYVSDLSRKRYGTLNRKYLLYFWYLIIISIFYGLPAVQLIMTYQRAVFETGNEDLCYYNFECAHSLGIFTAFNNIISNIGYVMLGLLFLGLTARRDILHRRTKNVNPNSQVLGIPQHYGLFYAMGLALTMEGLMSACYHMCPNFSNFQFDTAYMYILAMLIMLKIYQTRHPDVNASAHSAYMVMAVVIFLGVLGVLYGNQIFWIIFTIFFLIMSVVLTVEIYYMGQWNIDLCLPRRIYHLIRTDGIGCFRPTYLERMLLLLIANLVNFTLAGYGIVKRPRDFSTFLLSIFMINLLMYTFFYVIMKLRHRERFQMLSLVYILLACVSWGCAIYFYLTRTTTWEVTPAKSRALNQPCVLLDFYDAHDVWHFLSSVSMFFSFMLLMYLDDDLSKRPRNQIFVF

SEQ ID NO: 3 - protein, 954 aa, Schistosoma japonicum
  sjsid-b, SID-1 related B
MVFVLFGTKFTVACLIFKIYSVICEADLDKPYYGEVSQDQKTEYQFSLSSRSEYVIRVHVVNYNPKSAYPILVVIKQVDNVMSFQVPMVLNSISVYGNVSRTLCPIKLLPGEVRNLTVELSSAVEPSKRVRYLFLAQLVRDFDLESGVERNMLVSPAEPVYLRYLYPPGKNSAEIKVISKSDICMVLSIQKLQCPVNDLSDTVGNTGLHQTVTTLGAISIDVTQVFKGFFIVLVLKPTDYACSGIENIIPPLPDGGPLSLEPRVNLPGSRIKSVKILVTSAPRRWPYLLPILGAVGIYLLFYVVTIILILLYHRAERRKNFHDVSYDNPVIYNPTKVSLESLKKMKSAKDKKKSALASNSNIHQTSSDSLNVLRPSHTDTHHHILSLPQDYQSISLSNSLESNLHFRASIHQTPLVEIHGSHGWFSSTDDEDDYREDGCVCGTNNKIESRKKNRYFTGLNISTTSVDTNTTTGIILDNKQLPTNSAYSTMIGQETSHTPKRSVDLHPSNATNNANNSNDKHRNDKSDNVSSVSLTDEQTDVNSKFIPMYKMNVLLYVSDLSRKRYGTLNRKYLLYFWYLIIISIFYGLPAVQLIMTYQKAVFETGNEDLCYYNFECAHSLGIFTAFNNIISNIGYVMLGLLFLGLTARRDILHRRTKNVNPNSQVLGIPQHYGLFYAMGLALTMEGLMSACYHMCPNFSNFQFDTAYMYILAMLIMLKIYQTRHPDVNASAHSAYMVMAVVIFLGVLGVLYGNQIFWIIFTIFFLIMSVVLTVEIYYMGQWNIDLCLPRRIYHLIRTDGIGCFRPTYLERMLLLLIANLVNFTLAGYGIVKRPRDFSTFLLSIFMINLLMYTFFYVIMKLRHRERFQMLSLVYILLACVSWGCAIYFYLTRTTTWEVTPAKSRALNQPCVLLDFYDAHDVWHFLSSVSMFFSFMLLMYLDDDLSKRPRNQIFVF

SEQ ID NO: 4 - protein, 1055 aa, Schistosoma japonicum
  sjsid-a, SID-1 related A
MVFVLFGTKFTVACLIFKIYSVICEADLDKPYYGEVSQDQKTEYQFSLSSRSEYVIRVHVVNYNPKSAYPILVVIKQVDNVMSFQVPMVLNSISVYGNVSRTLCPIKLLPGEVRNLTVELSSAVEPSKRVRYLFLAQLVRDFDLESGVERNMLVSPAEPVYLRYLYPPGKNSAEIKVISKSDICMVLSIQKLQCPVNDLSDTVGNTGLHQTVTTLGAISIDVTQVFKGFFIVLVLKPTDYACSGIENIIPPLPDGGPLSLEPRVNLPGSRIKSVKILVTSAPRRWPYLLPILGAVGIYLLFYVVTIILILLYHRAERRKNFHDELMANFECGSDYPTVYSDNLRDSNVYHVNNTTISSTIPIQTATCSTSTSRSAGSQITTNLVNRRDRYNYGSLISSTSHHTKLHHHSVKSNSLQTQMIPVESVSYDNPVIYNPTKVSLESLKKMKSAKDKKKSALASNSNIHQTSSDSLNVLRPSHTDTHHHILSLPQDYQSISLSNSLESNLHFRASIHQTPLVEIHGSHGWFSSTDDEDDYREDGCVCGTNNKIESRKKNRYFTGLNISTTSVDTNTTTGIILDNKQLPTNSAYSTMIGQETSHTPKRSVDLHPSNATNNANNSNDKHRNDKSDNVSSVSLTDEQTDVNSKFIPMYKMNVLLYVSDLSRKRYGTLNRKYLLYFWYLIIISIFYGLPAVQLIMTYQKAVFETGNEDLCYYNFECAHSLGIFTAFNNIISNIGYVMLGLLFLGLTARRDILHRRTKNVNPNSQVLGIPQHYGLFYAMGLALTMEGLMSACYHMCPNFSNFQFDTAYMYILAMLIMLKIYQTRHPDVNASAHSAYMVMAVVIFLGVLGVLYGNQIFWIIFTIFFLIMSVVLTVEIYYMGQWNIDLCLPRRIYHLIRTDGIGCFRPTYLERMLLLLIANLVNFTLAGYGIVKRPRDFSTFLLSIFMINLLMYTFFYVIMKLRHRERFQMLSLVYILLACVSWGCAIYFYLTRTTTWEVTPAKSRALNQPCVLLDFYDAHDVWHFLSSVSMFFSFMLLMYLDDDLSKRPRNQIFVF

SEQ ID NO: 5 - protein, 877 aa, Bombyx mori
  silkworm sid-1-related gene3 (Sir-3)
MISWCALALCVSVVLASNITVEQRILNLEEEYTLVVTPSIEFILQFVPNEDQAEFPSRLWVRSVGGDTSRPLLLTARTKTGATTWQLPYQSGSMLMSELERTLCWDGSPTDAVGAPSECEGAGSQRGFTLHLASACAAPLTVTLRAAPARDWLLGFQARTTVTATQTGPAVNYYDFIPGQNSVRLIVESEDEVCATISVQRYTCPLAETIEDIDLTTLRMTVMRSGAVQLSRSLYPMGFYVVSLVRPDDAACSGEPAPEDDWLLEAALWAHTDRPSPPATLRQKTFTLTVRASLSRAQYMVGAGVTVAVFLLFYAGFAALVLAQRWPACARLTAPRAVLADAHKSESGALSEGVSVTGVTAETGVTSVTGVTSVTGVTSDAGTPVRTARRRRGSDATFDSSDASDTDSEEESPAVTNDTITNNMIANPTASSSAANPTTSPGTPGNHGAASPPDRANGAVTEGDAIERSTVQEETSRPFGLPARLHVAALARRGRRVLRARSDRYLHTLYTVAVFYALPVLQFVAAFQVMLNISGSLDMCYYNFLCAHPAGGLSDFNHVFSNLGYLLLGALFMLQLQRRKRNRKRAPRHEEYGIPAHYGLLSSLGAAMMVVALLSASYHVCPNSLNFQFDTAFMYVLAVLCMVKIYQSRHPDINARAHATFGVLAVFIALVVWGVLGGGPLFWSVFTVLHVFTFLLLSLRIYYVGQFRLEKSSLAVAARGLRARPLYTPRLVMLLIANAANWGFAIYGLLTHAGDIATHLLNVLLCNTLLYIVFYVLMKLLHGERIRWYSWCFLAAAAACWVPALYFFTSGSTDWSATPARSRHRNHECRVLQFYDSHDLWHMLSAAALYFTFNVMLTWDDGLSAVKRTEIAVFELI

SEQ ID NO: 6 - protein, 781 aa, Bombyx mori
  silkworm sid-1-related gene1 (Sir-1)
MMGYRKILLLMLIKISYCFKNSVNLAVNRTFQYNIYNYDTWINLQVNNTIEQILDFTEDSDKLLGFPTRVHVTTNSTLTSDHPLFITATQQKGVSSWELPLVLQTDDYFLMLNDMGRTLCPHDAGSDIRRESPPTVQLTTSSSANVSVDIKLKRVEDFYIELGKVNEVIVNPSSPRYYYFSFDQNPWNVSHAAGGPLDGTQRYNYNIPKSVILVIESDDEICATVSIQNNSCPVFDNEREVKYKGYHLTMSSQGGITLTQAMFPSGFYVVLIVRQSDADCTGASETEDAPKSFPAKRSKTFRLKIIATISYQEYLVGALVSAALVLLVALFVLALLLPCPCRCTEEVTVVVEESSPSTSREDSAETDTQPILEAGAADESWSREHALTVGKLTRAPPDTLARRSDRYFWGALTLAVVYALPVVQLLLTYQRMVFQTGDQDLCYYNFLCAHPLGTLSDFNHVFSNVGYVLLGAVFAGQVRFRQVKSRQRPENLGIPQHYGLLYSMGLALSMEGLLSACYHLCPNKMNFQFDSSFMYVIAVLVTLKLYQNRHSDIIPSAHSTFMILAVIMTIGLFGILHPSAGFAASFTLLHLGACLVLTLKIYYAGRFKMDRRVLLRAYAHVAARGWRSLLPAHPYRAGLLGLANLANWSLAGYSVYSHHNTDLARQLLAILMGNAILYTMFYMVMKLVNRERILARTWMYCILAHVAWFLALRLFLDSKTKWSETPAQSRQHNAPCSSLSFYDTHDLWHGVSAAALFLSFNMLLTMDDALRDTPRDQIPTF

SEQ ID NO: 7 - protein, 768 aa, Tribolium castaneum
  Sid-1-related C (Sirc)
MTPKMLHLFLIMSAVTVICDSFNPIYLNLSYSNFYTFSINKSVEYILEFSAPELKYPPRVTINSSDAQIKTPLMVVARQPKELLSWQLPMVLESDTGNHNFTKISRTLCHDMYRDYASRGITVDSPIVSVSTAAPRNVTFTVQVDYQKDFFIKPSVKYNFNITPSEPRFYFYNFTANITESPNSNYETVILEVFSDDFVCMTVSIQNASCLVFDTNQDITFRGFYETVNTQGGITIPKYKFPYGFFAVFVAKPDDSDCTGIPSLYYDTNRTKTITLIVKPSISYQDYVNAVIATLSSIGIFYFVLIAGFIFCSKRGYVPRQMEYVSSEPATPSTCLGEEVDEISLDETEYDVVSEADQDKSIRLGKSVVYLSDLARKDPRVHKYKSYLYLYNVLTVALFYGLPVIQLVVTYQRALNETGQQDLCYYNFLCAHPLGVISDFNHVFSNSGYVLLGLLFLGITYRREITHKDLNFERQYGIPQHYGMFYAMGVALIMEGVLSGSYHVCPNTANFQFDSSFMYVMAVLCMVKLYQNRHPDINATAYATFGVLAVAILLGMIGILEGNLYFWIVFTIIYLLSCFYLSIQIYYMGCWKLDAGLAMRVWRICVYEFWSGPLNVIKPIHKARMCLLIIANLCNWGMAFWGVYKHQKDFALFLLAIFMGNTLLYFSFYIVMKIINKERVNKLSLFFLSLSVLCAISAMYFFLNKSISWSRTPAQSRQFNQECKLLRFYDFHDIWHFLSAIGMFFTFMVLLTLDDDLSHTHRNKIVVF

SEQ ID NO: 8 - protein, 732 aa, Tribolium castaneum
  Sid-1-related B (Sirb)
MATSWFFVAIVPLVLCLQPKIVMVPQFGRVSQVMDFTLNSNIKYLLLYHPQNNNNPYSIKAWSDSASPQNPILIVVNQGIDTLSWSVPYSIFSQSEVYYHTSRTLCDSHNQNFTITLSTSAPTNTKLSMIVEEERFFHLVNGKRHTIEISPSEPRYFSYDYVPQSHSSLVTIEIDSDDETCLMVSVQKHTCPVLDLNNFINYQGFHQTILTKGGMRIRKKYYTGGFFLVFTVVEDEVCKKKDLPIIPNQNQSSTVHFTVTENIESKNHYIPAVFIVLACFILFSFVAIAIFCVFERYRKKKIAKNTEQIAMNVDEKTEEEIHEERDENNQQIPNNVADFSQNTQKNQKRSMNYLWQILNVGLFYIIPVIQLVVTLQSFLIQTGDFDLCYYNFRCANPLWIISDFNHVFSNIGYILMGIVFSINVFYRHFYSPPLTTGVPANYGVFYAMGAALIMEGVLSGCYHLCPNETNFQFDTSFMYVMIVLCLVKLYQNRHPDVTPTAYTTFSILGATILCGTIGIVFKAPPVFIVFVTIAYLVLLIYASLNIYHFGTARNFLRRCCLRNSEVPRPIQSPNTHRWWLLLLAITVNILLYGLGLILFYHTKTIDFATFILQILAGNAFLYTVVYTCMKIKCTSVRECTCSEKICAQAIIYGFLALVTWVLAGVFFFTEASKWTESPAQSRQLNKQCIFADFYDSRDLWHFFSSLALYFTFMYLLCIDDNLYTNRADIPLF

SEQ ID NO: 9 - protein, 757 aa, Tribolium castaneum
  Sid-1-related A (Sira)
MIAAAGLLLLVPLADCAHIASLNIEQHQGNYSQVMPFLFNQTTEHVLVFPTSDSIYPYRVKAWSSGAKLASPVLVVVRQEREVISWQVPFVVDTTMKDGVVHFHNTSRTLCHNDMPRIAKAKATSRILPIQLSQNFIIALSTSSLANVDISVMVEEERDFYLQEGRPYEVSVSPSESKYYYYKFHDKKNTSAMIEINSDDDVCLTVSIQDSFCPVFDLDKDITYEGKYQTINRKGGMTIRQREFPDGFFLVFVAKADNYQCSQKHSVLLVEHRKQHLILANRTSTITFTINKGINGKEYEIASLATLGALLSFCIVSTIMIFAFTRWGTISKFRPSGDELDADWEEPPEPPITRELKHELLSRQALTVNLLARAPEKDKRRSYNYLWHILSIAIFYSIPVVQLVITYQRVVNRTGDQDMCYYNFLCANPAFGLSDFNHIFSNVGYIIVGILFLGVVLHRQTKIPNSSTGIPVHYGVYYAMGIALIIEGILSACYHICPSQSNYQFDTSFMYVMAVLCMIKLYQNRHPDVNATAYATFTVLGMAIFLAMIGILNGSLTVWIVFVVIYSLLCAYISFKIYFISFVFDGFKQLKQSLKSSNKVEAIAPIRKSRFALLVIANIINYAMLITGLCLYNTGVTDFGTFLLGLLMGNSVLYAVFYTGMKLVNGERICFEAIIYGLLAIAAWATAAVYFLDNATLWTVTPAESRQWNQECIVMSFYDKHDVWHLLSAPALYLTFMFLLSLDDDLVDIKREEITVF

SEQ ID NO: 10 - protein, 827 aa, Homo sapiens
  human SID1 transmembrane family, member 1 (SIDT1), SID-1
MRGCLRLALLCALPWLLLAASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 11 - protein, 832 aa, Homo sapiens
  human SID1 transmembrane family, member 1 (SIDT1) (cDNA clone MGC150831 IMAGE 40125773), SID-1
MRGCLRLALLCALPWLLLAASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDVSDTDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 12 - protein, 776 aa, Caenorhabditis elegans; identical to SEQ ID NO: 1
  systemic RNAi enabling protein SID-1 (sid-1)

SEQ ID NO: 13 - protein, 535 aa, Zea mays
  sister of indeterminate spikelet 1 (sid1)
MASPAIPFAPLTSHRAAPFVLGCPPPWPPPPPPAARPRPPPPRPDAAAAARLLEEEAGAGSSRARSPGGPELESMVLDLNAESPTPGSASAASSSSVVVGGGFFRFDLLGGTPDEEGCSPSPPIVTRQLFPLPYPDAAGSTAASTASNGSPPPEVAGAWARRPADLGAPALAQGKVMSAPSSPAVLSPAAGKKSRRGPRSRSSQYRGVTFYRRTGRWESHIWDCGKQVYLGGFDTAHAAARAYDRAAIKFRGLDADINFQLKDYEDDLKQMRNWTKEEFVHILRRQSTGFARGSSKYRGVTLHKCGRWEARMGQLLGKKYIYLGLFDSEIEAARAYDRAAIRFNGPDAVRNFDSVSYDGDVPLPPAIEKDAVVDGDILDLNLRISQPNVHDLRSDGTLTGFGLSCNSPEASSSIVSQPMGPQWPVHPHSRSMRPQHPHLYASPCPGFFVNLREAPMQEEENRSEPACPQPFPSWAWQTQGSRAPVLPATTAASSGFSTAAATGVDAATAGHSVPPPSGSLRQFSGYHQLRFPPTA

SEQ ID NO: 14 - protein, 369 aa, Artificial Sequence
  SID-1 consensus sequence
DXXCXXNXXCAXXXXXXXXXNXXXXNXNXXXXNXXFXXXXXXRXXXXXXXXXXXXXXXXXXGXXXXXXXXXXXGXXXXXXXXXSXXYHXCPXXXXXQFDXXXXXXXXXLXXXXXXXXRHXXXXXXAXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXNXXXXXXXXXXXXXXXXXXXXXXXXXXXXXNXXXYXXXYXXMKXXXXXXXXXXXXEXXXXXXXXXXXXXXXXXXXAXXXXXXXXXXWXXXXAXSRXXNXXCXXXXFXXXXDXWHXXXXXXXXXXFXXXXXXDDXLXXXXXXXIXXF

SEQ ID NO: 15 - protein, 311 aa, Caenorhabditis elegans
  systemic RNAi enabling protein SID-2
MPRFVYFCFALIALLPISWTMDGILITDVEIHVDVCQISCKASNTASLLINDAPFTPMCNSAGDQIFFTYNGTAAISDLKNVTFILEVTTDTKNCTFTANYTGYFTPDPKSKPFQLGFASATLNRDMGKVTKTIMEDSGEMVEQDFSNSSAVPTPASTTPLPQSTVAHLTIAYVHLQYEETKTVVNKNGGAVAVAVIEGIALIAILAFLGYRTMVNHKLQNSTRTNGLYGYDNNNSSRITVPDAMRMSDIPPPRDPMYASPPTPLSQPTPARNTVMTTQELVVPTANSSAAQPSTTSNGQFNDPFATLESW

SEQ ID NO: 16 - protein, 302 aa, Caenorhabditis remanei
  systemic RNAi enabling protein SID-2
MIRYQTLVFAVFLLPVFWCFDSFLITSIEIRNDVGNINCTSSNLTINELALKPLCQIEEDANTKISYVTLTYNETESIPNGKNITFNLESSVTVKNYEPSQNMTNSANYQFMGIFVPDKSSKANTVLVRNVTLNKVEAPATTSASKFSEADVPISNKTILTVTYIHIQYDDSTKKEGNSNGGAVAVAIIEGIALIAILAYMGYRTMVKHRMKESTMNAALYGYDNNSRNSIRMSDIPPPRDPTYATPPTPTVTQQTPTRNTVMTTQELVVPPTQNTSAPAPTRPTTGASGQFNDPFDSLDSW

SEQ ID NO: 17 - protein, 314 aa, Caenorhabditis briggsae
  hypothetical protein CBG18280, SID-2
MIRNQILIIALFLIPVYWCIDVILISSIEVRNDVGSIDCTNSKLMINNQNFTPICEVGYDNTKSISYITLAYNASNSVQEGNTTYHLDTKVTVPNGNKTKTDDYQYTGVFVVDKTVQPNTVAVGYLTLEKFIPATTAAPPTTKPKKREAGFPQEQLDAEPTAPVSNKTSLTINYIRLKYEETSKQSNSNGGAVAVAIIEGIALIAILAYMGYRTMVKHRMKESSVNAAMYGFDNNSRNSIRMNDIPPPRDPTYATPPPAPFSQQPPARNTVMTTQELVVPQTSASVTRPTTTSNTTSNTTNGQFNDPFDSLDSW

SEQ ID NO: 18 - protein, 311 aa, Caenorhabditis elegans
  systemic RNAi enabling protein SID-2
MPRFVYFCFALIALLPISWTMDGILITDVEIHVDVCQISCKASNTASLLINDAPFTPMCNSAGDQIFFTYNGTAAISDLKNVTFILEVTTDTKNCTFTANYTGYFTPDPKSKPFQLGFASATLNRDMGKVTKTIMEDSGEMVEQDFSNSSAVPTPASTTPLPQSTVAHLTIAYVHLQYEETKTVVNKNGGAVAVAVIEGIALIAILAFGGYRTMVNHKLQNSTRTNGLYGYDNNNSSRIRVPDAMRMSDIPPPRDPMYASPPTPLSQPTPARNTVMTTQELVVPTANSSAAQPSTTSNGQFNDPFATLESW

SEQ ID NO: 19 - protein, 311 aa, Caenorhabditis elegans; identical to SEQ ID NO: 15
  Systemic RNA Interference Defective family member (sid-2)

SEQ ID NO: 20 - protein, 311 aa, Caenorhabditis elegans; identical to SEQ ID NO: 15
  protein ZK520.2, SID-2

SEQ ID NO: 21 - protein, 334 aa, Artificial Sequence
  SID-2 consensus sequence
MXRXXXXXXAXXXLXPXXWXXDXXLIXXXEXXXDVXXIXCXXSXXXXLXINXXXXXPXCXXXXDXXXXXXXXXXXYNXXXXXXXXXNXTXXLXXXXXXXNXXXXXXXTXXXXXXXXGXFXXDXXXXXXXXXXXXXTLXXXXXXXXXXXXXXXXXXXXXXFXXXXXXXXXXXXXXXPXSXXXXLTXXYXXXXYXXXXXXXXNXNGGAVAVAXIEGIALIAILAXXGYRTMVXHXXXXSXXXXXXYGXDNNXXXXXXXXXXXRMXDIPPPRDPXYAXPPXXXXXQXXPXRNTVMTTQELVVPXXXXXXXXPXXTXXXXXXXXXGQFNDPFXXLXSW

SEQ ID NO: 22 - protein, 234 aa, Artificial Sequence
  His-tagged MSP1E1
MGHHHHHHIEGRLKLLDNWDSVTSTFSKLREQLGPVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 23 - protein, 256 aa, Artificial Sequence
  His-tagged MSP1E2
MGHHHHHHIEGRLKLLDNWDSVTSTFSKLREQLGPVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 24 - protein, 278 aa, Artificial Sequence
  His-tagged MSP1E3
MGHHHHHHIEGRLKLLDNWDSVTSTFSKLREQLGPVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 25 - protein, 223 aa, Artificial Sequence [sequence omitted]

SEQ ID NO: 26 - protein, 200 aa, Artificial Sequence
  MSP1NH
LKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 27 - protein, 212 aa, Artificial Sequence
  His-tagged MSP1T2
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 28 - protein, 189 aa, Artificial Sequence
  MSP1T2NH
STFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 29 - protein, 201 aa, Artificial Sequence
  MSP1T3
MGHHHHHHHDYDIPTTENLYFQGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 30 - protein, 168 aa, Artificial Sequence
  MSP1D4D5
MGHHHHHHIEGRLKLLDNWDSVTSTFSKLREQLGPVTQEFWDNLEKETEGLRQEMSKDLEEVKAKVQPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 31 - protein, 168 aa, Artificial Sequence
  His-tagged MSP1D6D7
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSARLAEYHAKATEHLSTLSEKAKPALEDLRQGLLPVLESFKVSFLSALEEYTKKLNTQ

SEQ ID NO: 32 - protein, 190 aa, Artificial Sequence
  His-tagged MSP1D3D9
MGHHHHHHIEGRLKLLDNWDSVTSTFSKLREQLGPVTQEFWDNLEKETEGLRQEMSPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPVLESFKVSFLSALEEYTKKLNTQ

SEQ ID NO: 33 - protein, 201 aa, Artificial Sequence
  His-tagged MSP1D10.5
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLSALEEYTKKLNTQ

SEQ ID NO: 34 - protein, 190 aa, Artificial Sequence
  His-tagged MSP1D3D10.5
MGHHHHHHIEGRLKLLDNWDSVTSTFSKLREQLGPVTQEFWDNLEKETEGLRQEMSPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLSALEEYTKKLNTQ

SEQ ID NO: 35 - protein, 381 aa, Artificial Sequence
  His-tagged MSP2D1D1
MGHHHHHHHDYDIPTTENLYFQGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 36 - protein, 262 aa, Danio rerio
  zebrafish ZAP-1 apolipoprotein
MKFVALALTLLLALGSQANLFQADAPTQLEHYKAAALVYLNQVKDQAEKALDNLDGTDYEQYKLQLSESLTKLQEYAQTTSQALTPYAETISTQLMENTKQLRERVMTDVEDLRSKLEPHRAELYTALQKHIDEYREKLEPVFQEYSALNRQNAEQLRAKLEPLMDDIRKAFESNIEETKSKVVPMVEAVRTKLTERLEDLRTMAAPYAEEYKEQLVKAVEEAREKIAPHTQDLQTRMEPYMENVRTTFAQMYETIAKAIQA

SEQ ID NO: 37 - protein, 285 aa, Bombyx mori
  silkmoth apolipoprotein
MWRLTVLVLAATASAQIPSLGWCPDFQSMANFNMNRFLGTWYEAERFFTVSELGSRCVTTNYVSTPEGRIIVSNEIVNSLTGMKRLMEGSLQMIGREGEGRFMIKYSSLPLPYESEFSILDTDYDNYAVMWSCSGIGPVHTQNTWLLTRERLPSLMAMQNAYAVLDRFKISRTFFVKTNQADCTILPDPVAIPIEAKSADVIKNVDIKVKEKEPVEDSDSVKKQIIDEVVQERSAVPEISFEPKPVPVPEMILTENEKKGENMEEPKAEDKAEAVEPKAVETTTI

SEQ ID NO: 38 - protein, 317 aa, Homo sapiens [sequence omitted]

SEQ ID NO: 39 - protein, 267 aa, Homo sapiens
  human APO A-1 apolipoprotein
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

(19) United States
(12) Patent Application Publication
(10) Pub. No.: US 2011/0177599 A1
(43) Pub. Date: Jul. 21, 2011

(54) DELIVERY OF NUCLEIC ACIDS ACROSS MEMBRANES

(75) Inventor: Camille Diges, Concord, CA (US)

(73) Assignee: Bio-Rad Laboratories, Inc., Hercules, CA (US)

(21) Appl. No.: 12/840,115

(22) Filed: Jul. 20, 2010

Related U.S. Application Data
(60) Provisional application No. 61/227,008, filed on Jul. 20, 2009.

(51) Int. Cl. C12N 15/00; C12N 5/00; C07K 14/00

(52) U.S. Cl. 435/455; 435/325; 435/346; 530/359

(57) ABSTRACT

The present invention provides for methods and compositions for introducing integral membrane proteins into cell membranes and, optionally, delivery of nucleic acids across membranes via the integral membrane proteins.

### CROSS-REFERENCE TO RELATED PATENT APPLICATIONS

The present patent application claims benefit of priority to U.S. Provisional Patent Application No. 61/227,008, filed Jul. 20, 2009, which is incorporated by reference.

### BACKGROUND OF THE INVENTION

Typically, when one desires to introduce a protein into a cell, one transfects the cell with a corresponding nucleic acid encoding the protein. Upon proper introduction into the cell, and with appropriate conditions, a transfected cell will express the desired protein. The present invention overcomes the need to transfect cells where it is desired to introduce an integral membrane protein into the cell.

### BRIEF SUMMARY OF THE INVENTION

The present invention provides for isolated cells comprising a cell membrane, the membrane comprising an integral membrane polypeptide, wherein the integral membrane polypeptide is heterologous to the cell and the cell does not comprise a nucleic acid encoding the polypeptide, or wherein the membrane comprises copies of the integral membrane polypeptide that were not translated in the cell.

In some embodiments, the cell does not comprise a nucleic acid encoding the polypeptide. In some embodiments, the membrane comprises copies of the integral membrane polypeptide that were not translated in the cell.

In some embodiments, the integral membrane polypeptide is a nucleic acid transporter polypeptide. In some embodiments, the nucleic acid transporter polypeptide is an RNA transporter. In some embodiments, the nucleic acid transporter polypeptide comprises an amino acid sequence at least 80% identical to any of SEQ ID NOs:1-21. In some embodiments, the nucleic acid transporter polypeptide comprises any of SEQ ID NOs:1-21.

In some embodiments, the cell is mammalian cell.

The present invention also provides for methods of making the cell described above or elsewhere herein. In some embodiments, the method comprises contacting a cell having a cell membrane with an apolipoprotein bound lipid bilayer comprising the integral membrane polypeptide under conditions to allow for fusion of the lipid bilayer to the cell membrane, thereby introducing the polypeptide into the cell membrane.

In some embodiments, the integral membrane protein is a nucleic acid transporter polypeptide. In some embodiments, the nucleic acid transporter polypeptide is an RNA transporter. In some embodiments, the nucleic acid transporter polypeptide comprises an amino acid sequence at least 80% identical to any of SEQ ID NOs:1-21. In some embodiments, the nucleic acid transporter polypeptide comprises any of SEQ ID NOs:1-21.

In some embodiments, the contacting step comprising contacting the cell with an agent that enhances fusion in the presence of the lipid bilayer. In some embodiments, the agent is selected from the group consisting of polyethylene glycol (PEG), dimethyl sulfoxide (DMSO), pyrene butyrate, and phosphate buffered saline either with or without supplementary divalent and monvalent salts. For example, the supplementary salts can be calcium and magnesium salts.

The present invention also provides for methods of introducing an exogenous nucleic acid into a cells comprising a cell membrane, wherein the membrane comprising a nucleic acid transporter polypeptide. In some embodiments, the method comprises contacting the cells with an exogenous nucleic acid, thereby introducing the exogenous nucleic acid into the cell.

In some embodiments, the polypeptide is an RNA transporter and the exogenous nucleic acid comprises RNA. In some embodiments, the polypeptide is an RNA transporter and the exogenous nucleic acid comprises double stranded RNA. In some embodiments, the polypeptide is an RNA transporter and the exogenous nucleic acid is an siRNA.

The present invention also provides apolipoprotein bound lipid bilayer comprising a nucleic acid transporter polypeptide. In some embodiments, the nucleic acid transporter polypeptide is an RNA transporter. In some embodiments, the nucleic acid transporter polypeptide comprises an amino acid sequence at least 80% identical to any of SEQ ID NOs:1-21. In some embodiments, the nucleic acid transporter polypeptide comprises any of SEQ ID NOs:1-21.

Additional aspects of the invention will be clear from a review of the remainder of this document.

### DEFINITIONS

A “nucleic acid transporter” protein, as used herein, refers to an integral membrane protein that allows for passage of a nucleic acid across a membrane. Some nucleic acid transporters allow for transport of only some types of nucleic acid (e.g., RNA, not DNA, etc.). Methods for measuring nucleic acid transport into cells are readily available and are known in the art.

The term apolipoprotein bound lipid bilayer refers to lipid bilayers, often discoidal in shape, bound by lipoproteins. Apolipoprotein bound lipid bilayer are discoidal in shape and are one phospholipid bilayer thick. Generation of apolipoprotein bound lipid bilayers generally results in a relatively uniform sized structures. In some embodiments, the diameter of the structures are between 5-15 nm, e.g., between 8-12 nm, e.g., 10 nm in diameter.

The term “isolated,” when applied to a nucleic acid or protein, denotes that the nucleic acid or protein is essentially free of other cellular components with which it is associated in the natural state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” The present invention provides for polypeptides comprising an amino acid sequence substantially identical to any of SEQ ID NOs: 1-35. Identity is determined across the entire length of the test sequence (e.g., a sequence listened in a claim), unless where indicated. Optionally, the identity exists over a region that is at least about 50 amino acids or nucleotides in length, or over a region that is 75-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Exogenous,” refers to any polynucleotide, polypeptide or protein sequence, whether chimeric or not, that is initially or subsequently introduced into the genome of an individual host cell or the organism regenerated from said host cell by any means other than by inheritance through cell division. Examples of means by which this can be accomplished include, for example, introduction by recombinant gene expression or introduction into a cell membrane by a apolipoprotein bound lipid bilayer. The term “exogenous” as used herein is also intended to encompass inserting a naturally found element into a non-naturally found location.

A “heterologous” protein, when in reference to a cell, indicates that the protein is not naturally produced by, or present in or on, the cell.

### DETAILED DESCRIPTION

### I. Introduction

The present invention provides for novel methods of introducing integral membrane proteins into cells without transfection of the cells with polynucleotides encoding the proteins. For example, an apolipoprotein bound lipid bilayer comprising the desired integral membrane protein(s) can be fused with a cell, thereby releasing the integral membrane protein into the cell membrane of the cell.

In some embodiments, the integral membrane protein is a nucleic acid transporter. By introducing a nucleic acid transporter into the cell membrane of a cell, one can facilitate delivery of nucleic acids into cells. This is of particular use for cells that are recalcitrant to nucleic acid transfection because the methods of the invention do not require transfection with a nucleic acid encoding the nucleic acid transporter. Such methods are also of particular use for delivery of siRNAs, dsRNAs, and other RNAs (e.g., microRNAs, shRNAs, etc.) that regulate gene expression in the cell or are otherwise of interest or benefit.

### II. Integral Membrane Proteins

Any integral membrane protein that can be inserted into a apolipoprotein bound lipid bilayer can be used according to the methods of the invention. Integral membrane proteins can include for example proteins having one or more transmembrane region. Many integral membrane proteins contain residues with hydrophobic side chains that interact with fatty acyl groups of the membrane phospholipids, thus anchoring the protein to the membrane. Many integral proteins span the entire phospholipid bilayer. These transmembrane proteins contain one or more membrane-spanning domains, and optionally, domains (e.g., from 1-100 s of amino acids long) extending into the aqueous medium on one or both sides of the lipid membrane bilayer.

Integral membrane proteins have a wide variety of functions and as such it can be desirable to introduce such proteins into any of a number of cell types, including but not limited to, cell types for which it is difficult or not technically possible to transform. Once the integral membrane protein is introduced into the cell, the activity of the protein in the cell can be monitored, tested, or otherwise used as appropriate. Exemplary integral membrane proteins include but are not limited to nucleic acid transporters, ion channels (e.g., voltage-gated receptors, including but not limited to sodium, potassium, and calcium channels, and ligand gated channels, including but not limited to NAD and ATP receptors) and G-protein coupled receptors (e.g., beta-adrenergic receptors, chemokine receptors including but not limited to CCR5 and serotonin receptor).

A. Nucleic Acid Transporters

In some embodiments, the integral membrane protein is a nucleic acid transporter. Such proteins are useful, for example, when in the membrane of a cell, for introduction of nucleic acids into the cells via the transporter.

In some embodiments, the nucleic acid transporter is an DNA transporter protein (including but not limited to a DNA transporter of bacterial origin, e.g., such as Com EA, Com EC or any other DNA translocase). In some embodiments, the nucleic acid transporter is an RNA transporter protein. In some embodiments, the transporter is capable of transporting single-stranded and/or double-stranded RNA. For example, in some embodiments the nucleic acid transporter is SID-1 or SID-2 or is a homolog or ortholog thereof. Translocation of nucleic acids across cellular membranes is associated with the viral infection, bacterial conjugation, and transport of nuclear encoded tRNAs into mitochondria and SID-1 proteins are involved with these translocation processes. The first gene characterized, SID-1, encodes a transmembrane protein expressed in all cells sensitive to systemic RNAi and appears to be involved in cellular spreading of dsRNA. SID-1 and SID-2 was originally identified as an RNA transporter in C. elegans and has been found to mediate RNA, including siRNA, transport into cells that express the protein. See, e.g., Feinberg and Hunter, Science 301:1545-1547 (2003) and WO 2004/099386.

SID-1 proteins are predicted to have 9 transmembrane domains based on sequence analysis, that passively transport dsRNA in a concentration-dependent, ATP-independent fashion. SID-1 proteins can generally transport dsRNAs of at least between 15-1000 nucleotides in length. A variety of SID-1 proteins are known in the art. A number of such proteins are set forth in Table 1. The present invention provides for SID-1 polypeptides that are substantially similar to any of SEQ ID NO:s 1-13. The present invention also provides for polypeptides comprising the SID-1 consensus sequence, SEQ ID NO:14, as listed in Table 1. Additional consensus sequences and alignments are available for example, in Dong and Friedrich, BMC Biotechnology 5(25) (2005) and supplementary figures therein.

SID-2 is transmembrane protein expressed in the intestine and localized in the apical (luminal) membrane in Caenorhabditis species. The gene sid-2 is specifically required for the uptake of silencing information (for example, dsRNA) from the environment. SID-2 is sufficient to confer environment RNAi on the RNAi defective species, Caenorhabditis briggsae. SID-2 appears to be involved with dsRNA uptake from the environment. See, e.g., Winston et al., Proc. Natl. Acad Sci. USA 104(25):10565-70 (2007).

A variety of SID-2 proteins are known in the art. A number of such proteins are set forth in Table 2. The present invention provides for SID-2 polypeptides that are substantially similar to any of SEQ ID NO:s 15-20. The present invention also provides for polypeptides comprising the SID-1 consensus sequence, SEQ ID NO:21, as listed in Table 2.

TABLE 1
SID-1 Homologs

| GenBank | SEQ | | |
|---|---|---|---|
| Accession | ID | Description | |
| No. | NO: | [Organism] | Amino Acid Sequence |
| NM_071971 | 1 | Systemic RNA | MIRVYLIILMHLVIGLTQNNSTTPSPIITSSNSSVLV |
| | | Interference | FEISSKMKMIEKKLEANTVHVLRLELDQSFILDLTKV |
| | | Defective family | AAEIVDSSKYSKEDGVILEVTVSNGRDSFLLKLPTVY |
| | | member (sid-1) | PNLKLYTDGKLLNPLVEQDFGAHRKRHRIGDPHFHQN |
| | | (sid-1) mRNA, | LIVTVQSRLNADIDYRLHVTHLDRAQYDFLKFKTGQT |
| | | complete cds | TKTLSNQKLTFVKPIGFFLNCSEQNISQFHVTLYSED |
| | | [Caenorhabditis | DICANLITVPANESIYDRSVISDKTHNRRVLSFTKRA |
| | | elegans] | DIFFTETEISMFKSFRIFVFIAPDDSGCSTNTSRKSF |
| | | | NEKKKISFEFKKLENQSYAVPTALMMIFLTTPCLLFL |
| | | | PIVINIIKNSRKLAPSQSNLISFSPVPSEQRDMDLSH |
| | | | DEQQNTSSELENNGEIPAAENQIVEEITAENQETSVE |
| | | | EGNREIQVKIPLKQDSLSLHGQMLQYPVAIILPVLMH |
| | | | TAIEFHKWTTSTMANRDEMCFHNHACARPLGELRAWN |
| | | | NIITNIGYTLYGAIFIVLSICRRGRHEYSHVFGTYEC |
| | | | TLLDVTIGVFMVLQSIASATYHICPSDVAFQFDTPCI |
| | | | QVICGLLMVRQWFVRHESPSPAYTNILLVGVVSLNFL |
| | | | ISAFSKTSYVRFIIAVIHVIVVGSICLAKERSLGSEK |
| | | | LKTRFFIMAFSMGNFAAIVMYLTLSAFHLNQIATYCF |
| | | | IINCIMYLMYYGCMKVLHSERITSKAKLCGALSLLAW |
| | | | AVAGFFFFQDDTDWTRSAAASRALNKPCLLLGFFGSH |
| | | | DLWHIFGALAGLFTFIFVSFVDDDLINTRKTSINIF |
| AB480305 | 2 | sjsid-c mRNA for | MESRKKNRYFTGLNISTTSVDTNTTTGIILDNKQLPT |
| | | SID-1 related C, | SSAYSTMIGQETSHTPKRSVDLHPSNATNNANNSNDK |
| | | complete cds | HRNDKSDNVSSVSLTDEQTDINSKFIPMYKMNVLLYV |
| | | [Schistosoma | SDLSRKRYGTLNRKYLLYFWYLIIISIFYGLPAVQLI |
| | | japonicum] | MTYQRAVFETGNEDLCYYNFECAHSLGIFTAFNNIIS |
| | | | NIGYVMLGLLFLGLTARRDILHRRTKNVNPNSQVLGI |
| | | | PQHYGLFYAMGLALTMEGLMSACYHMCPNFSNFQFDT |
| | | | AYMYILAMLIMLKIYQTRHPDVNASAHSAYMVMAVVI |
| | | | FLGVLGVLYGNQIFWIIFTIFFLIMSVVLTVEIYYMG |
| | | | QWNIDLCLPRRIYHLIRTDGIGCFRPTYLERMLLLLI |
| | | | ANLVNFTLAGYGIVKRPRDFSTFLLSIFMINLLMYTF |
| | | | FYVIMKLRHRERFQMLSLVYILLACVSWGCAIYFYLT |
| | | | RTTTWEVTPAKSRALNQPCVLLDFYDAHDVWHFLSSV |
| | | | SMFFSFMLLMYLDDDLSKRPRNQIFVF |
| AB480304 | 3 | sjsid-b mRNA for | MVFVLFGTKFTVACLIFKIYSVICEADLDKPYYGEVS |
| | | SID-1 related B, | QDQKTEYQFSLSSRSEYVIRVHVVNYNPKSAYPILVV |
| | | complete | IKQVDNVMSFQVPMVLNSISVYGNVSRTLCPIKLLPG |
| | | cds | EVRNLTVELSSAVEPSKRVRYLFLAQLVRDFDLESGV |
| | | [Schistosoma | ERNMLVSPAEPVYLRYLYPPGKNSAEIKVISKSDICM |
| | | japonicum] | VLSIQKLQCPVNDLSDTVGNTGLHQTVTTLGAISIDV |
| | | | TQVFKGFFIVLVLKPTDYACSGIENIIPPLPDGGPLS |
| | | | LEPRVNLPGSRIKSVKILVTSAPRRWPYLLPILGAVG |
| | | | IYLLFYVVTIILILLYHRAERRKNFHDVSYDNPVIYN |
| | | | PTKVSLESLKKMKSAKDKKKSALASNSNIHQTSSDSL |
| | | | NVLRPSHTDTHHHILSLPQDYQSISLSNSLESNLHFR |
| | | | ASIHQTPLVEIHGSHGWFSSTDDEDDYREDGCVCGTN |
| | | | NKIESRKKNRYFTGLNISTTSVDTNTTTGIILDNKQL |
| | | | PTNSAYSTMIGQETSHTPKRSVDLHPSNATNNANNSN |
| | | | DKHRNDKSDNVSSVSLTDEQTDVNSKFIPMYKMNVLL |
| | | | YVSDLSRKRYGTLNRKYLLYFWYLIIISIFYGLPAVQ |
| | | | LIMTYQKAVFETGNEDLCYYNFECAHSLGIFTAFNNI |
| | | | ISNIGYVMLGLLFLGLTARRDILHRRTKNVNPNSQVL |
| | | | GIPQHYGLFYAMGLALTMEGLMSACYHMCPNFSNFQF |
| | | | DTAYMYILAMLIMLKIYQTRHPDVNASAHSAYMVMAV |
| | | | VIFLGVLGVLYGNQIFWIIFTIFFLIMSVVLTVEIYY |
| | | | MGQWNIDLCLPRRIYHLIRTDGIGCFRPTYLERMLLL |
| | | | LIANLVNFTLAGYGIVKRPRDFSTFLLSIFMINLLMY |
| | | | TFFYVIMKLRHRERFQMLSLVYILLACVSWGCAIYFY |
| | | | LTRTTTWEVTPAKSRALNQPCVLLDFYDAHDVWHFLS |
| | | | SVSMFFSFMLLMYLDDDLSKRPRNQIFVF |
| AB480303 | 4 | sjsid-a mRNA for | MVFVLFGTKFTVACLIFKIYSVICEADLDKPYYGEVS |
| | | SID-1 related A, | QDQKTEYQFSLSSRSEYVIRVHVVNYNPKSAYPILVV |
| | | complete cds | IKQVDNVMSFQVPMVLNSISVYGNVSRTLCPIKLLPG |
| | | [Schistosoma | EVRNLTVELSSAVEPSKRVRYLFLAQLVRDFDLESGV |
| | | japonicum] | ERNMLVSPAEPVYLRYLYPPGKNSAEIKVISKSDICM |
| | | | VLSIQKLQCPVNDLSDTVGNTGLHQTVTTLGAISIDV |
| | | | TQVFKGFFIVLVLKPTDYACSGIENIIPPLPDGGPLS |
| | | | LEPRVNLPGSRIKSVKILVTSAPRRWPYLLPILGAVG |
| | | | IYLLFYVVTIILILLYHRAERRKNFHDELMANFECGS |
| | | | DYPTVYSDNLRDSNVYHVNNTTISSTIPIQTATCSTS |
| | | | TSRSAGSQITTNLVNRRDRYNYGSLISSTSHHTKLHH |
| | | | HSVKSNSLQTQMIPVESVSYDNPVIYNPTKVSLESLK |
| | | | KMKSAKDKKKSALASNSNIHQTSSDSLNVLRPSHTDT |
| | | | HHHILSLPQDYQSISLSNSLESNLHFRASIHQTPLVE |
| | | | IHGSHGWFSSTDDEDDYREDGCVCGTNNKIESRKKNR |
| | | | YFTGLNISTTSVDTNTTTGIILDNKQLPTNSAYSTMI |
| | | | GQETSHTPKRSVDLHPSNATNNANNSNDKHRNDKSDN |
| | | | VSSVSLTDEQTDVNSKFIPMYKMNVLLYVSDLSRKRY |
| | | | GTLNRKYLLYFWYLIIISIFYGLPAVQLIMTYQKAVF |
| | | | ETGNEDLCYYNFECAHSLGIFTAFNNIISNIGYVMLG |
| | | | LLFLGLTARRDILHRRTKNVNPNSQVLGIPQHYGLFY |
| | | | AMGLALTMEGLMSACYHMCPNFSNFQFDTAYMYILAM |
| | | | LIMLKIYQTRHPDVNASAHSAYMVMAVVIFLGVLGVL |
| | | | YGNQIFWIIFTIFFLIMSVVLTVEIYYMGQWNIDLCL |
| | | | PRRIYHLIRTDGIGCFRPTYLERMLLLLIANLVNFTL |
| | | | AGYGIVKRPRDFSTFLLSIFMINLLMYTFFYVIMKLR |
| | | | HRERFQMLSLVYILLACVSWGCAIYFYLTRTTTWEVT |
| | | | PAKSRALNQPCVLLDFYDAHDVWHFLSSVSMFFSFML |
| | | | LMYLDDDLSKRPRNQIFVF |
| NM_001113 | 5 | sid-1-related gene3 | MISWCALALCVSVVLASNITVEQRILNLEEEYTLVVT |
| 265 | | (Sir-3), mRNA | PSIEFILQFVPNEDQAEFPSRLWVRSVGGDTSRPLLL |
| | | [Bombyx mori] | TARTKTGATTWQLPYQSGSMLMSELERTLCWDGSPTD |
| | | | AVGAPSECEGAGSQRGFTLHLASACAAPLTVTLRAAP |
| | | | ARDWLLGFQARTTVTATQTGPAVNYYDFIPGQNSVRL |
| | | | IVESEDEVCATISVQRYTCPLAETIEDIDLTTLRMTV |
| | | | MRSGAVQLSRSLYPMGFYVVSLVRPDDAACSGEPAPE |
| | | | DDWLLEAALWAHTDRPSPPATLRQKTFTLTVRASLSR |
| | | | AQYMVGAGVTVAVFLLFYAGFAALVLAQRWPACARLT |
| | | | APRAVLADAHKSESGALSEGVSVTGVTAETGVTSVTG |
| | | | VTSVTGVTSDAGTPVRTARRRRGSDATFDSSDASDTD |
| | | | SEEESPAVTNDTITNNMIANPTASSSAANPTTSPGTP |
| | | | GNHGAASPPDRANGAVTEGDAIERSTVQEETSRPFGL |
| | | | PARLHVAALARRGRRVLRARSDRYLHTLYTVAVFYAL |
| | | | PVLQFVAAFQVMLNISGSLDMCYYNFLCAHPAGGLSD |
| | | | FNHVFSNLGYLLLGALFMLQLQRRKRNRKRAPRHEEY |
| | | | GIPAHYGLLSSLGAAMMVVALLSASYHVCPNSLNFQF |
| | | | DTAFMYVLAVLCMVKIYQSRHPDINARAHATFGVLAV |
| | | | FIALVVWGVLGGGPLFWSVFTVLHVFTFLLLSLRIYY |
| | | | VGQFRLEKSSLAVAARGLRARPLYTPRLVMLLIANAA |
| | | | NWGFAIYGLLTHAGDIATHLLNVLLCNTLLYIVFYVL |
| | | | MKLLHGERIRWYSWCFLAAAAACWVPALYFFTSGSTD |
| | | | WSATPARSRHRNHECRVLQFYDSHDLWHMLSAAALYF |
| | | | TFNVMLTWDDGLSAVKRTEIAVFELI |
| NM_001113 | 6 | sid-1-related gene1 | MMGYRKILLLMLIKISYCFKNSVNLAVNRTFQYNIYN |
| 264 | | (Sir-1), mRNA | YDTWINLQVNNTIEQILDFTEDSDKLLGFPTRVHVTT |
| | | [Bombyx mori] | NSTLTSDHPLFITATQQKGVSSWELPLVLQTDDYFLM |
| | | | LNDMGRTLCPHDAGSDIRRESPPTVQLTTSSSANVSV |
| | | | DIKLKRVEDFYIELGKVNEVIVNPSSPRYYYFSFDQN |
| | | | PWNVSHAAGGPLDGTQRYNYNIPKSVILVIESDDEIC |
| | | | ATVSIQNNSCPVFDNEREVKYKGYHLTMSSQGGITLT |
| | | | QAMFPSGFYVVLIVRQSDADCTGASETEDAPKSFPAK |
| | | | RSKTFRLKIIATISYQEYLVGALVSAALVLLVALFVL |
| | | | ALLLPCPCRCTEEVTVVVEESSPSTSREDSAETDTQP |
| | | | ILEAGAADESWSREHALTVGKLTRAPPDTLARRSDRY |
| | | | FWGALTLAVVYALPVVQLLLTYQRMVFQTGDQDLCYY |
| | | | NFLCAHPLGTLSDFNHVFSNVGYVLLGAVFAGQVRFR |
| | | | QVKSRQRPENLGIPQHYGLLYSMGLALSMEGLLSACY |
| | | | HLCPNKMNFQFDSSFMYVIAVLVTLKLYQNRHSDIIP |
| | | | SAHSTFMILAVIMTIGLFGILHPSAGFAASFTLLHLG |
| | | | ACLVLTLKIYYAGRFKMDRRVLLRAYAHVAARGWRSL |
| | | | LPAHPYRAGLLGLANLANWSLAGYSVYSHHNTDLARQ |
| | | | LLAILMGNAILYTMFYMVMKLVNRERILARTWMYCIL |
| | | | AHVAWFLALRLFLDSKTKWSETPAQSRQHNAPCSSLS |
| | | | FYDTHDLWHGVSAAALFLSFNMLLTMDDALRDTPRDQ |
| | | | IPTF |
| NM_001105 | 7 | Sid-1-related C | MTPKMLHLFLIMSAVTVICDSFNPIYLNLSYSNFYTF |
| 658 | | (Sirc), mRNA | SINKSVEYILEFSAPELKYPPRVTINSSDAQIKTPLM |
| | | [Tribolium | VVARQPKELLSWQLPMVLESDTGNHNFTKISRTLCHD |
| | | castaneum] | MYRDYASRGITVDSPIVSVSTAAPRNVTFTVQVDYQK |
| | | | DFFIKPSVKYNFNITPSEPRFYFYNFTANITESPNSN |
| | | | YETVILEVFSDDFVCMTVSIQNASCLVFDTNQDITFR |
| | | | GFYETVNTQGGITIPKYKFPYGFFAVFVAKPDDSDCT |
| | | | GIPSLYYDTNRTKTITLIVKPSISYQDYVNAVIATLS |
| | | | SIGIFYFVLIAGFIFCSKRGYVPRQMEYVSSEPATPS |
| | | | TCLGEEVDEISLDETEYDVVSEADQDKSIRLGKSVVY |
| | | | LSDLARKDPRVHKYKSYLYLYNVLTVALFYGLPVIQL |
| | | | VVTYQRALNETGQQDLCYYNFLCAHPLGVISDFNHVF |
| | | | SNSGYVLLGLLFLGITYRREITHKDLNFERQYGIPQH |
| | | | YGMFYAMGVALIMEGVLSGSYHVCPNTANFQFDSSFM |
| | | | YVMAVLCMVKLYQNRHPDINATAYATFGVLAVAILLG |
| | | | MIGILEGNLYFWIVFTIIYLLSCFYLSIQIYYMGCWK |
| | | | LDAGLAMRVWRICVYEFWSGPLNVIKPIHKARMCLLI |
| | | | IANLCNWGMAFWGVYKHQKDFALFLLAIFMGNTLLYF |
| | | | SFYIVMKIINKERVNKLSLFFLSLSVLCAISAMYFFL |
| | | | NKSISWSRTPAQSRQFNQECKLLRFYDFHDIWHFLSA |
| | | | IGMFFTFMVLLTLDDDLSHTHRNKIVVF |
| NM_001109 | 8 | Sid-1-related B | MATSWFFVAIVPLVLCLQPKIVMVPQFGRVSQVMDFT |
| 783 | | (Sirb), mRNA | LNSNIKYLLLYHPQNNNNPYSIKAWSDSASPQNPILI |
| | | [Tribolium | VVNQGIDTLSWSVPYSIFSQSEVYYHTSRTLCDSHNQ |
| | | castaneum] | NFTITLSTSAPTNTKLSMIVEEERFFHLVNGKRHTIE |
| | | | ISPSEPRYFSYDYVPQSHSSLVTIEIDSDDETCLMVS |
| | | | VQKHTCPVLDLNNFINYQGFHQTILTKGGMRIRKKYY |
| | | | TGGFFLVFTVVEDEVCKKKDLPIIPNQNQSSTVHFTV |
| | | | TENIESKNHYIPAVFIVLACFILFSFVAIAIFCVFER |
| | | | YRKKKIAKNTEQIAMNVDEKTEEEIHEERDENNQQIP |
| | | | NNVADFSQNTQKNQKRSMNYLWQILNVGLFYIIPVIQ |
| | | | LVVTLQSFLIQTGDFDLCYYNFRCANPLWIISDFNHV |
| | | | FSNIGYILMGIVFSINVFYRHFYSPPLTTGVPANYGV |
| | | | FYAMGAALIMEGVLSGCYHLCPNETNFQFDTSFMYVM |
| | | | IVLCLVKLYQNRHPDVTPTAYTTFSILGATILCGTIG |
| | | | IVFKAPPVFIVFVTIAYLVLLIYASLNIYHFGTARNF |
| | | | LRRCCLRNSEVPRPIQSPNTHRWWLLLLAITVNILLY |
| | | | GLGLILFYHTKTIDFATFILQILAGNAFLYTVVYTCM |
| | | | KIKCTSVRECTCSEKICAQAIIYGFLALVTWVLAGVF |
| | | | FFTEASKWTESPAQSRQLNKQCIFADFYDSRDLWHFF |
| | | | SSLALYFTFMYLLCIDDNLYTNRADIPLF |
| NM_001105 | 9 | Sid-1-related A | MIAAAGLLLLVPLADCAHIASLNIEQHQGNYSQVMPF |
| 542 | | (Sira), mRNA | LFNQTTEHVLVFPTSDSIYPYRVKAWSSGAKLASPVL |
| | | [Tribolium | VVVRQEREVISWQVPFVVDTTMKDGVVHFHNTSRTLC |
| | | castaneum] | HNDMPRIAKAKATSRILPIQLSQNFIIALSTSSLANV |
| | | | DISVMVEEERDFYLQEGRPYEVSVSPSESKYYYYKFH |
| | | | DKKNTSAMIEINSDDDVCLTVSIQDSFCPVFDLDKDI |
| | | | TYEGKYQTINRKGGMTIRQREFPDGFFLVFVAKADNY |
| | | | QCSQKHSVLLVEHRKQHLILANRTSTITFTINKGING |
| | | | KEYEIASLATLGALLSFCIVSTIMIFAFTRWGTISKF |
| | | | RPSGDELDADWEEPPEPPITRELKHELLSRQALTVNL |
| | | | LARAPEKDKRRSYNYLWHILSIAIFYSIPVVQLVITY |
| | | | QRVVNRTGDQDMCYYNFLCANPAFGLSDFNHIFSNVG |
| | | | YIIVGILFLGVVLHRQTKIPNSSTGIPVHYGVYYAMG |
| | | | IALIIEGILSACYHICPSQSNYQFDTSFMYVMAVLCM |
| | | | IKLYQNRHPDVNATAYATFTVLGMAIFLAMIGILNGS |
| | | | LTVWIVFVVIYSLLCAYISFKIYFISFVFDGFKQLKQ |
| | | | SLKSSNKVEAIAPIRKSRFALLVIANIINYAMLITGL |
| | | | CLYNTGVTDFGTFLLGLLMGNSVLYAVFYTGMKLVNG |
| | | | ERICFEAIIYGLLAIAAWATAAVYFLDNATLWTVTPA |
| | | | ESRQWNQECIVMSFYDKHDVWHLLSAPALYLTFMFLL |
| | | | SLDDDLVDIKREEITVF |
| NM_017699 | 10 | SID1 transmembrane | MRGCLRLALLCALPWLLLAASPGHPAKSPRQPPAPRR |
| | | family, member 1 | DPFDAARGADFDHVYSGVVNLSTENIYSFNYTSQPDQ |
| | | (SIDT1), mRNA | VTAVRVYVNSSSENLNYPVLVVVRQQKEVLSWQVPLL |
| | | [Homo sapiens] | FQGLYQRSYNYQEVSRTLCPSEATNETGPLQQLIFVD |
| | | | VASMAPLGAQYKLLVTKLKHFQLRTNVAFHFTASPSQ |
| | | | PQYFLYKFPKDVDSVIIKVVSEMAYPCSVVSVQNIMC |
| | | | PVYDLDHNVEFNGVYQSMTKKAAITLQKKDFPGEQFF |
| | | | VVFVIKPEDYACGGSFFIQEKENQTWNLQRKKNLEVT |
| | | | IVPSIKESVYVKSSLFSVFIFLSFYLGCLLVGFVHYL |
| | | | RFQRKSIDGSFGSNDGSGNMVASHPIAASTPEGSNYG |
| | | | TIDESSSSPGRQMSSSDGGPPGQSDTDSSVEESDFDT |
| | | | MPDIESDKNIIRTKMFLYLSDLSRKDRRIVSKKYKIY |
| | | | FWNIITIAVFYALPVIQLVITYQTVVNVTGNQDICYY |
| | | | NFLCAHPLGVLSAFNNILSNLGHVLLGFLFLLIVLRR |
| | | | DILHRRALEAKDIFAVEYGIPKHFGLFYAMGIALMME |
| | | | GVLSACYHVCPNYSNFQFDTSFMYMIAGLCMLKLYQT |
| | | | RHPDINASAYSAYASFAVVIMVTVLGVVFGKNDVWFW |
| | | | VIFSAIHVLASLALSTQIYYMGRFKIDLGIFRRAAMV |
| | | | FYTDCIQQCSRPLYMDRMVLLVVGNLVNWSFALFGLI |
| | | | YRPRDFASYMLGIFICNLLLYLAFYIIMKLRSSEKVL |
| | | | PVPLFCIVATAVMWAAALYFFFQNLSSWEGTPAESRE |
| | | | KNRECILLDFFDDHDIWHFLSATALFFSFLVLLTLDD |
| | | | DLDVVRRDQIPVF |
| BC117222 | 11 | SID1 transmembrane | MRGCLRLALLCALPWLLLAASPGHPAKSPRQPPAPRR |
| | | family, member 1, | DPFDAARGADFDHVYSGVVNLSTENIYSFNYTSQPDQ |
| | | mRNA (cDNA clone | VTAVRVYVNSSSENLNYPVLVVVRQQKEVLSWQVPLL |
| | | MGC: 150831 | FQGLYQRSYNYQEVSRTLCPSEATNETGPLQQLIFVD |
| | | IMAGE: 40125773), | VASMAPLGAQYKLLVTKLKHFQLRTNVAFHFTASPSQ |
| | | complete cds | PQYFLYKFPKDVDSVIIKVVSEMAYPCSVVSVQNIMC |
| | | [Homo sapiens] | PVYDLDHNVEFNGVYQSMTKKAAITLQKKDFPGEQFF |
| | | | VVFVIKPEDYACGGSFFIQEKENQTWNLQRKKNLEVT |
| | | | IVPSIKESVYVKSSLFSVFIFLSFYLGCLLVGFVHYL |
| | | | RFQRKSIDGSFGSNDGSGNMVASHPIAASTPEGSNYG |
| | | | TIDESSSSPGRQMSSSDGGPPGQSDTDSSVEESDFDT |
| | | | MPDIESDKNIIRTKMFLYLSDLSRKDRRIVSKKYKIY |
| | | | FWNIITIAVFYALPVIQLVITYQTVVNVTGNQDICYY |
| | | | NFLCAHPLGVLSAFNNILSNLGHVLLGFLFLLIVLRR |
| | | | DILHRRALEAKDIFAVEYGIPKHFGLFYAMGIALMME |
| | | | GVLSACYHVCPNYSNFQFDTSFMYMIAGLCMLKLYQT |
| | | | RHPDINASAYSAYASFAVVIMVTVLGVVFGKNDVWFW |
| | | | VIFSAIHVLASLALSTQIYYMGRFKIDVSDTDLGIFR |
| | | | RAAMVFYTDCIQQCSRPLYMDRMVLLVVGNLVNWSFA |
| | | | LFGLIYRPRDFASYMLGIFICNLLLYLAFYIIMKLRS |
| | | | SEKVLPVPLFCIVATAVMWAAALYFFFQNLSSWEGTP |
| | | | AESREKNRECILLDFFDDHDIWHFLSATALFFSFLVL |
| | | | LTLDDDLDVVRRDQIPVF |
| AF478687 | 12 | systemic RNAi | MIRVYLIILMHLVIGLTQNNSTTPSPIITSSNSSVLV |
| | | enabling protein | FEISSKMKMIEKKLEANTVHVLRLELDQSFILDLTKV |
| | | SID-1 (sid-1) mRNA, | AAEIVDSSKYSKEDGVILEVTVSNGRDSFLLKLPTVY |
| | | complete cds | PNLKLYTDGKLLNPLVEQDFGAHRKRHRIGDPHFHQN |
| | | [Caenorhabditis | LIVTVQSRLNADIDYRLHVTHLDRAQYDFLKFKTGQT |
| | | elegans] | TKTLSNQKLTFVKPIGFFLNCSEQNISQFHVTLYSED |
| | | | DICANLITVPANESIYDRSVISDKTHNRRVLSFTKRA |
| | | | DIFFTETEISMFKSFRIFVFIAPDDSGCSTNTSRKSF |
| | | | NEKKKISFEFKKLENQSYAVPTALMMIFLTTPCLLFL |
| | | | PIVINIIKNSRKLAPSQSNLISFSPVPSEQRDMDLSH |
| | | | DEQQNTSSELENNGEIPAAENQIVEEITAENQETSVE |
| | | | EGNREIQVKIPLKQDSLSLHGQMLQYPVAIILPVLMH |
| | | | TAIEFHKWTTSTMANRDEMCFHNHACARPLGELRAWN |
| | | | NIITNIGYTLYGAIFIVLSICRRGRHEYSHVFGTYEC |
| | | | TLLDVTIGVFMVLQSIASATYHICPSDVAFQFDTPCI |
| | | | QVICGLLMVRQWFVRHESPSPAYTNILLVGVVSLNFL |
| | | | ISAFSKTSYVRFIIAVIHVIVVGSICLAKERSLGSEK |
| | | | LKTRFFIMAFSMGNFAAIVMYLTLSAFHLNQIATYCF |
| | | | IINCIMYLMYYGCMKVLHSERITSKAKLCGALSLLAW |
| | | | AVAGFFFFQDDTDWTRSAAASRALNKPCLLLGFFGSH |
| | | | DLWHIFGALAGLFTFIFVSFVDDDLINTRKTSINIF |
| NP00113953 | 13 | sister of | MASPAIPFAPLTSHRAAPFVLGCPPPWPPPPPPAAR |
| 9 | | indeterminate | PRPPPPRPDAAAAARLLEEEAGAGSSRARSPGGPEL |
| | | spikelet 1 (sid1) | ESMVLDLNAESPTPGSASAASSSSVVVGGGFFRFDL |
| | | [Zea mays] | LGGTPDEEGCSPSPPIVTRQLFPLPYPDAAGSTAAS |
| | | | TASNGSPPPEVAGAWARRPADLGAPALAQGKVMSAP |
| | | | SSPAVLSPAAGKKSRRGPRSRSSQYRGVTFYRRTGR |
| | | | WESHIWDCGKQVYLGGFDTAHAAARAYDRAAIKFRG |
| | | | LDADINFQLKDYEDDLKQMRNWTKEEFVHILRRQST |
| | | | GFARGSSKYRGVTLHKCGRWEARMGQLLGKKYIYLG |
| | | | LFDSEIEAARAYDRAAIRFNGPDAVRNFDSVSYDGD |
| | | | VPLPPAIEKDAVVDGDILDLNLRISQPNVHDLRSDG |
| | | | TLTGFGLSCNSPEASSSIVSQPMGPQWPVHPHSRSM |
| | | | RPQHPHLYASPCPGFFVNLREAPMQEEENRSEPACP |
| | | | QPFPSWAWQTQGSRAPVLPATTAASSGFSTAAATGV |
| | | | DAATAGHSVPPPSGSLRQFSGYHQLRFPPTA |
| SID-1 | 14 | SID-1 Consensus | DXXCXXNXXCAXXXXXXXXXNXXXXNXNXXXXNXXF |
| Consensus | | Sequence | XXXXXXRXXXXXXXXXXXXXXXXXXGXXXXXXXXXX |
| Sequence | | | XGXXXXXXXXXSXXYHXCPXXXXXQFDXXXXXXXXX |
| | | | LXXXXXXXXRHXXXXXXAXXXXXXXXXXXXXXXXXX |
| | | | XXXXXXXXXXXXXXXXXXXXXXXXXXXXXXXXXXXX |
| | | | XXXXXXXXXXXXXXXXXXXXXXXXXXXXXXXXXXXX |
| | | | XXXXXXXNXXXXXXXXXXXXXXXXXXXXXXXXXXXX |
| | | | XNXXXYXXXYXXMKXXXXXXXXXXXXEXXXXXXXXX |
| | | | XXXXXXXXXXAXXXXXXXXXXWXXXXAXSRXXNXXC |
| | | | XXXXFXXXXDXWHXXXXXXXXXXFXXXXXXDDXLXX |
| | | | XXXXXIXXF |

TABLE 2
SID-2 Homologs

| GenBank | SEQ | | |
|---|---|---|---|
| Accession | ID | Description | |
| No. | NO: | [Organism] | Amino Acid Sequence |
| AAS45709 | 15 | SID-2 | MPRFVYFCFALIALLPISWTMDGILITDVEIHVDVC |
| | | [Caenorhabditis | QISCKASNTASLLINDAPFTPMCNSAGDQIFFTY |
| | | elegans] | NGTAAISDLKNVTFILEVTTDTKNCTFTANYTGYFT |
| | | | PDPKSKPFQLGFASATLNRDMGKVTKTIMEDSGE |
| | | | MVEQDFSNSSAVPTPASTTPLPQSTVAHLTIAYVHL |
| | | | QYEETKTVVNKNGGAVAVAVIEGIALIAILAFLG |
| | | | YRTMVNHKLQNSTRTNGLYGYDNNNSSRITVPDAMR |
| | | | MSDIPPPRDPMYASPPTPLSQPTPARNTVMTTQE |
| | | | LVVPTANSSAAQPSTTSNGQFNDPFATLESW |
| N/A | 16 | SID-2 | MIRYQTLVFAVFLLPVFWCFDSFLITSIEIRNDVGN |
| | | [Caenorhabditis | INCTSSNLTINELALKPLCQIEEDANTKISYVTLTY |
| | | remanei] | NETESIPNGKNITFNLESSVTVKNYEPSQNMTNSAN |
| | | | YQFMGIFVPDKSSKANTVLVRNVTLNKVEAPATTSA |
| | | | SKFSEADVPISNKTILTVTYIHIQYDDSTKKEGNSN |
| | | | GGAVAVAIIEGIALIAILAYMGYRTMVKHRMKESTM |
| | | | NAALYGYDNNSRNSIRMSDIPPPRDPTYATPPTPTV |
| | | | TQQTPTRNTVMTTQELVVPPTQNTSAPAPTRPTTGA |
| | | | SGQFNDPFDSLDSW |
| XP001665880 | 17 | hypothetical | MIRNQILIIALFLIPVYWCIDVILISSIEVRNDVGS |
| | | protein CBG18280; | IDCTNSKLMINNQNFTPICEVGYDNTKSISYITLAY |
| | | SID-2 | NASNSVQEGNTTYHLDTKVTVPNGNKTKTDDYQYTG |
| | | [Caenorhabditis | VFVVDKTVQPNTVAVGYLTLEKFIPATTAAPPTTKP |
| | | briggsae] | KKREAGFPQEQLDAEPTAPVSNKTSLTINYIRLKYE |
| | | | ETSKQSNSNGGAVAVAIIEGIALIAILAYMGYRTMV |
| | | | KHRMKESSVNAAMYGFDNNSRNSIRMNDIPPPRDPT |
| | | | YATPPPAPFSQQPPARNTVMTTQELVVPQTSASVTR |
| | | | PTTTSNTTSNTTNGQFNDPFDSLDSW |
| N/A | 18 | SID-2 | MPRFVYFCFALIALLPISWTMDGILITDVEIHVDVC |
| | | [Caenorhabditis | QISCKASNTASLLINDAPFTPMCNSAGDQIFFTYNG |
| | | elegans] | TAAISDLKNVTFILEVTTDTKNCTFTANYTGYFTPD |
| | | | PKSKPFQLGFASATLNRDMGKVTKTIMEDSGEMVEQ |
| | | | DFSNSSAVPTPASTTPLPQSTVAHLTIAYVHLQYEE |
| | | | TKTVVNKNGGAVAVAVIEGIALIAILAFGGYRTMVN |
| | | | HKLQNSTRTNGLYGYDNNNSSRIRVPDAMRMSDIPP |
| | | | PRDPMYASPPTPLSQPTPARNTVMTTQELVVPTANS |
| | | | SAAQPSTTSNGQFNDPFATLESW |
| NP499823 | 19 | Systemic RNA | MPRFVYFCFALIALLPISWTMDGILITDVEIHVDVC |
| | | Interference | QISCKASNTASLLINDAPFTPMCNSAGDQIFFTY |
| | | Defective family | NGTAAISDLKNVTFILEVTTDTKNCTFTANYTGYFT |
| | | member (sid-2) | PDPKSKPFQLGFASATLNRDMGKVTKTIMEDSGE |
| | | [Caenorhabditis | MVEQDFSNSSAVPTPASTTPLPQSTVAHLTIAYVHL |
| | | elegans] | QYEETKTVVNKNGGAVAVAVIEGIALIAILAFLG |
| | | | YRTMVNHKLQNSTRTNGLYGYDNNNSSRITVPDAMR |
| | | | MSDIPPPRDPMYASPPTPLSQPTPARNTVMTTQE |
| | | | LVVPTANSSAAQPSTTSNGQFNDPFATLESW |
| CAB07300 | 20 | protein ZK520.2, | MPRFVYFCFALIALLPISWTMDGILITDVEIHVDVC |
| | | confirmed by | QISCKASNTASLLINDAPFTPMCNSAGDQIFFTY |
| | | transcript evidence | NGTAAISDLKNVTFILEVTTDTKNCTFTANYTGYFT |
| | | [Caenorhabditis | PDPKSKPFQLGFASATLNRDMGKVTKTIMEDSGE |
| | | elegans] | MVEQDFSNSSAVPTPASTTPLPQSTVAHLTIAYVHL |
| | | | QYEETKTVVNKNGGAVAVAVIEGIALIAILAFLG |
| | | | YRTMVNHKLQNSTRTNGLYGYDNNNSSRITVPDAMR |
| | | | MSDIPPPRDPMYASPPTPLSQPTPARNTVMTTQE |
| | | | LVVPTANSSAAQPSTTSNGQFNDPFATLESW |
| SID-2 | 21 | SID-2 Consensus | MXRXXXXXXAXXXLXPXXWXXDXXLIXXXEXXXDVX |
| Consensus | | Sequence | XIXCXXSXXXXLXINXXXXXPXCXXXXDXXXXXXXX |
| | | | XXXYNXXXXXXXXXNXTXXLXXXXXXXNXXXXXXXT |
| | | | XXXXXXXXGXFXXDXXXXXXXXXXXXXTLXXXXXXX |
| | | | XXXXXXXXXXXXXXXFXXXXXXXXXXXXXXXPXSXX |
| | | | XXLTXXYXXXXYXXXXXXXXNXNGGAVAVAXIEGIA |
| | | | LIAILAXXGYRTMVXHXXXXSXXXXXXYGXDNNXXX |
| | | | XXXXXXXXRMXDIPPPRDPXYAXPPXXXXXQXXPXR |
| | | | NTVMTTQELVVPXXXXXXXXPXXTXXXXXXXXXGQF |
| | | | NDPFXXLXSW |

### III. Apolipoprotein Bound Lipid Bilayer

The present invention provides for apolipoprotein bound lipid bilayers comprising an integral membrane protein (including but not limited to nucleic acid transporters as described herein) as well as their use to transfer the integral membrane proteins from the apolipoprotein bound lipid bilayers to cell membranes.

The present invention provides for use of any apolipoprotein bound lipid bilayer in the methods of the invention. Apolipoprotein bound lipid bilayers (some of which are also referred to as nanolipoprotein particles (NLPs), nanoscale apolipoprotein bound bilayers (NABBs) or nanodiscs in the scientific literature) are self-assembling discoidal non-cellular, non-liposomal particles composed of planar phospholipid membrane bilayers surrounded (i.e., “bound”) by a scaffold apolipoprotein. Apolipoprotein bound lipid bilayers are of a discrete size and shape (e.g., approximately 10 nm discs) that are reproducible and can be used to fold a predetermined number (e.g., 1, 2, or more) of proteins per particle. Without intending to be bound by a particular theory of action, it is believed that apolipoprotein bound lipid bilayer, in contrast for example to liposomes, will fuse with the cell membrane rather than be taken up via endocytosis. A number of scaffold proteins are known and have been described. See, e.g., U.S. Pat. Nos. 7,083,958 and 7,048,949; and Katzen et al., J. Proteome 7:3535-3542 (2008); Cappuccio et al., Mol. Cell. Proteomics 7:2246-2253 (2008); Banerjee et al., J. Mol. Biology 337(4): 1067-1081 (2008).

While it is not believed to be essential, a number of scaffold lipoproteins described in the literature are apolipoproteins or fragments or derivatives thereof. Apolipoproteins have proline-containing amphipathic alpha-helical domains that are able to associate with lipid acyl chains. The proline residues are thought to “kink” the helices to help the protein bend around the lipid to form a disc. In some embodiments, there are two apolipoproteins wrapping around the lipid bilayer to form a disc. The apolipoproteins that form these particles have a semi-conserved pattern of high hydrophobicity in the regions that are allow for formation of nanoparticles. Without intending to limit the scope of the invention, it is believed that apolipoproteins from any species, or modified versions thereof, can be used for the generation of apolipoprotein bound lipid bilayers. A variety of apolipoproteins and variants thereof have been described, for example, in Chromy et al., J. Am Chem. Soc. 129(46):14348-14534 (2007). Apolipoprotein bound lipid bilayers have been formed using for example, human (e.g., SEQ ID NO: 38-39), zebrafish (e.g., ZAP-1, e.g., SEQ ID NO:36)) and silk moth B. mori (e.g., SEQ ID NO: 37) apolipoproteins, or variants thereof, as scaffold proteins. See also, Banerjee, et al., J. Mol. Biol. 377:1067-1081 (2008). Accordingly, in some embodiments, the scaffold proteins are substantially identical to SEQ ID NOs: 36-39. In some embodiments, for example, the scaffold protein is substantially identical to the N-terminal 22 kD fragment of human apolipoprotein E4 (apoE422K) as described in Katzen et al., J. Proteome 7:3535-3542 (2008).

| SEQ | | |
|---|---|---|
| ID | | |
| NO: | Description | Amino Acid Sequence |
| 36 | ZAP-1 | MKFVALALTLLLALGSQANLFQADAPTQLEHYKAAALVYLNQVKDQAEKALDNLD |
| | | GTDYEQYKLQLSESLTKLQEYAQTTSQALTPYAETISTQLMENTKQLRERVMTDV |
| | | EDLRSKLEPHRAELYTALQKHIDEYREKLEPVFQEYSALNRQNAEQLRAKLEPLM |
| | | DDIRKAFESNIEETKSKVVPMVEAVRTKLTERLEDLRTMAAPYAEEYKEQLVKAV |
| | | EEAREKIAPHTQDLQTRMEPYMENVRTTFAQMYETIAKAIQA |
| 37 | silk moth | MWRLTVLVLAATASAQIPSLGWCPDFQSMANFNMNRFLGTWYEAERFFTVSELGS |
| | B. mori | RCVTTNYVSTPEGRIIVSNEIVNSLTGMKRLMEGSLQMIGREGEGRFMIKYSSLP |
| | apolipoprotein | LPYESEFSILDTDYDNYAVMWSCSGIGPVHTQNTWLLTRERLPSLMAMQNAYAVL |
| | | DRFKISRTFFVKTNQADCTILPDPVAIPIEAKSADVIKNVDIKVKEKEPVEDSDS |
| | | VKKQIIDEVVQERSAVPEISFEPKPVPVPEMILTENEKKGENMEEPKAEDKAEAV |
| | | EPKAVETTTI |
| 38 | APO E422K | MKVLWAALLVTFLAGCQAKVEQAVETEPEPELRQQTEWQSGQRWELALGRFWDYL |
| | | RWVQTLSEQVQEELLSSQVTQELRALMDETMKELKAYKSELEEQLTPVAEETRAR |
| | | LSKELQAAQARLGADMEDVCGRLVQYRGEVQAMLGQSTEELRVRLASHLRKLRKR |
| | | LLRDADDLQKRLAVYQAGAREGAERGLSAIRERLGPLVEQGRVRAATVGSLAGQP |
| | | LQERAQAWGERLRARMEEMGSRTRDRLDEVKEQVAEVRAKLEEQAQQIRLQAEAF |
| | | QARLKSWFEPLVEDMQRQWAGLVEKVQAAVGTSAAPVPSDNH |
| 39 | APO A-1 | MKAAVLTLAVLFLTGSQARHFWQQDEPPQSPWDRVKDLATVYVDVLKDSGRDYVS |
| | | QFEGSALGKQLNLKLLDNWDSVTSTFSKLREQLGPVTQEFWDNLEKETEGLRQEM |
| | | SKDLEEVKAKVQPYLDDFQKKWQEEMELYRQKVEPLRAELQEGARQKLHELQEKL |
| | | SPLGEEMRDRARAHVDALRTHLAPYSDELRQRLAARLEALKENGGARLAEYHAKA |
| | | TEHLSTLSEKAKPALEDLRQGLLPVLESFKVSFLSALEEYTKKLNTQ |

Various synthetic apolipoprotein sequences can also be used. For example, U.S. Pat. No. 7,083,958 describes an artificial variant referred to as “MSP2” comprising a tandem repeat of protein MSP1 with a short linker. These sequences as well as others disclosed in the '958 patent are provided herein as SEQ ID NOs: 22-35. The present invention provides for apolipoprotein bound lipid bilayers constructed using any scaffold protein that is substantially identical to any of SEQ ID NOs:22-35, including fusion proteins comprising such sequences. Fusion proteins include, e.g., tags including but not limited to poly-His tags that allow for purification of the proteins.

MSP Sequences From US7083958

| SEQ | | |
|---|---|---|
| ID | | |
| NO: | Description | Amino Acid Sequence |
| 22 | His-tagged MSP1E1 | MGHHHHHHIEGRLKLLDNWDSVTSTFSKLREQLGPVTQEFWDN |
| | | LEKETEGLRQEMSKDLEEVKAKVQPYLDDFQKKWQEEMELYRQ |
| | | KVEPYLDDFQKKWQEEMELYRQKVEPLRAELQEGARQKLHELQ |
| | | EKLSPLGEEMRDRARAHVDALRTHLAPYSDELRQRLAARLEAL |
| | | KENGGARLAEYHAKATEHLSTLSEKAKPALEDLRQGLLPVLES |
| | | FKVSFLSALEEYTKKLNTQ |
| 23 | His-tagged MSP1E2 | MGHHHHHHIEGRLKLLDNWDSVTSTFSKLREQLGPVTQEFWDN |
| | | LEKETEGLRQEMSKDLEEVKAKVQPYLDDFQKKWQEEMELYRQ |
| | | KVEPYLDDFQKKWQEEMELYRQKVEPLRAELQEGARQKLHELQ |
| | | EKLSPLRAELQEGARQKLHELQEKLSPLGEEMRDRARAHVDAL |
| | | RTHLAPYSDELRQRLAARLEALKENGGARLAEYHAKATEHLST |
| | | LSEKAKPALEDLRQGLLPVLESFKVSFLSALEEYTKKLNTQ |
| 24 | His-tagged MSP1E3 | MGHHHHHHIEGRLKLLDNWDSVTSTFSKLREQLGPVTQEFWDN |
| | | LEKETEGLRQEMSKDLEEVKAKVQPYLDDFQKKWQEEMELYRQ |
| | | KVEPYLDDFQKKWQEEMELYRQKVEPLRAELQEGARQKLHELQ |
| | | EKLSPLGEEMRDRARAHVDALRTHLAPLRAELQEGARQKLHEL |
| | | QEKLSPLGEEMRDRARAHVDALRTHLAPYSDELRQRLAARLEA |
| | | LKENGGARLAEYHAKATEHLSTLSEKAKPALEDLRQGLLPVLE |
| | | SFKVSFLSALEEYTKKLNTQ |
| 25 | His-tagged MSP1TEV | MGHHHHHHHDYDIPTTENLYFQGLKLLDNWDSVTSTFSKLREQ |
| | | LGPVTQEFWDNLEKETEGLRQEMSKDLEEVKAKVQPYLDDFQK |
| | | KWQEEMELYRQKVEPLRAELQEGARQKLHELQEKLSPLGEEMR |
| | | DRARAHVDALRTHLAPYSDELRQRLAARLEALKENGGARLAEY |
| | | HAKATEHLSTLSEKAKPALEDLRQGLLPVLESFKVSFLSALEE |
| | | YTKKLNTQ |
| 26 | MSP1NH | LKLLDNWDSVTSTFSKLREQLGPVTQEFWDNLEKETEGLRQEM |
| | | SKDLEEVKAKVQPYLDDFQKKWQEEMELYRQKVEPLRAELQEG |
| | | ARQKLHELQEKLSPLGEEMRDRARAHVDALRTHLAPYSDELRQ |
| | | RLAARLEALKENGGARLAEYHAKATEHLSTLSEKAKPALEDLR |
| | | QGLLPVLESFKVSFLSALEEYTKKLNTQ |
| 27 | His-tagged MSP1T2 | MGHHHHHHHDYDIPTTENLYFQGSTFSKLREQLGPVTQEFWDN |
| | | LEKETEGLRQEMSKDLEEVKAKVQPYLDDFQKKWQEEMELYRQ |
| | | KVEPLRAELQEGARQKLHELQEKLSPLGEEMRDRARAHVDALR |
| | | THLAPYSDELRQRLAARLEALKENGGARLAEYHAKATEHLSTL |
| | | SEKAKPALEDLRQGLLPVLESFKVSFLSALEEYTKKLNTQ |
| 28 | MSP1T2NH | STFSKLREQLGPVTQEFWDNLEKETEGLRQEMSKDLEEVKAKV |
| | | QPYLDDFQKKWQEEMELYRQKVEPLRAELQEGARQKLHELQEK |
| | | LSPLGEEMRDRARAHVDALRTHLAPYSDELRQRLAARLEALKE |
| | | NGGARLAEYHAKATEHLSTLSEKAKPALEDLRQGLLPVLESFK |
| | | VSFLSALEEYTKKLNTQ |
| 29 | MSP1T3 | MGHHHHHHHDYDIPTTENLYFQGPVTQEFWDNLEKETEGLRQE |
| | | MSKDLEEVKAKVQPYLDDFQKKWQEEMELYRQKVEPLRAELQE |
| | | GARQKLHELQEKLSPLGEEMRDRARAHVDALRTHLAPYSDELR |
| | | QRLAARLEALKENGGARLAEYHAKATEHLSTLSEKAKPALEDL |
| | | RQGLLPVLESFKVSFLSALEEYTKKLNTQ |
| 30 | MSP1D4D5 | MGHHHHHHIEGRLKLLDNWDSVTSTFSKLREQLGPVTQEFWDN |
| | | LEKETEGLRQEMSKDLEEVKAKVQPLGEEMRDRARAHVDALRT |
| | | HLAPYSDELRQRLAARLEALKENGGARLAEYHAKATEHLSTLS |
| | | EKAKPALEDLRQGLLPVLESFKVSFLSALEEYTKKLNTQ |
| 31 | His-tagged MSP1D6D7 | MGHHHHHHIEGRLKLLDNWDSVTSTFSKLREQLGPVTQEFWDN |
| | | LEKETEGLRQEMSKDLEEVKAKVQPYLDDFQKKWQEEMELYRQ |
| | | KVEPLRAELQEGARQKLHELQEKLSARLAEYHAKATEHLSTLS |
| | | EKAKPALEDLRQGLLPVLESFKVSFLSALEEYTKKLNTQ |
| 32 | His-tagged MSP1D3D9 | MGHHHHHHIEGRLKLLDNWDSVTSTFSKLREQLGPVTQEFWDN |
| | | LEKETEGLRQEMSPYLDDFQKKWQEEMELYRQKVEPLRAELQE |
| | | GARQKLHELQEKLSPLGEEMRDRARAHVDALRTHLAPYSDELR |
| | | QRLAARLEALKENGGARLAEYHAKATEHLSTLSEKAKPVLESF |
| | | KVSFLSALEEYTKKLNTQ |
| 33 | His-tagged MSP1D10.5 | MGHHHHHHIEGRLKLLDNWDSVTSTFSKLREQLGPVTQEFWDN |
| | | LEKETEGLRQEMSKDLEEVKAKVQPYLDDFQKKWQEEMELYRQ |
| | | KVEPLRAELQEGARQKLHELQEKLSPLGEEMRDRARAHVDALR |
| | | THLAPYSDELRQRLAARLEALKENGGARLAEYHAKATEHLSTL |
| | | SEKAKPALEDLRQGLLSALEEYTKKLNTQ |
| 34 | His-tagged MSP1D3D10.5 | MGHHHHHHIEGRLKLLDNWDSVTSTFSKLREQLGPVTQEFWDN |
| | | LEKETEGLRQEMSPYLDDFQKKWQEEMELYRQKVEPLRAELQE |
| | | GARQKLHELQEKLSPLGEEMRDRARAHVDALRTHLAPYSDELR |
| | | QRLAARLEALKENGGARLAEYHAKATEHLSTLSEKAKPALEDL |
| | | RQGLLSALEEYTKKLNTQ |
| 35 | His-tagged MSP2D1D1 | MGHHHHHHHDYDIPTTENLYFQGPVTQEFWDNLEKETEGLRQE |
| | | MSKDLEEVKAKVQPYLDDFQKKWQEEMELYRQKVEPLRAELQE |
| | | GARQKLHELQEKLSPLGEEMRDRARAHVDALRTHLAPYSDELR |
| | | QRLAARLEALKENGGARLAEYHAKATEHLSTLSEKAKPALEDL |
| | | RQGLLPVLESFKVSFLSALEEYTKKLNTQGTPVTQEFWDNLEK |
| | | ETEGLRQEMSKDLEEVKAKVQPYLDDFQKKWQEEMELYRQKVE |
| | | PLRAELQEGARQKLHELQEKLSPLGEEMRDRARAHVDALRTHL |
| | | APYSDELRQRLAARLEALKENGGARLAEYHAKATEHLSTLSEK |
| | | AKPALEDLRQGLLPVLESFKVSFLSALEEYTKKLNTQ |

Apolipoprotein bound lipid bilayers can be generated by any method known in the art. For example, in some embodiments, a scaffold protein, cholate, and 1,2-dimyrostoyl-sn-glycero-3-phosphocholine (DMPC) are mixed in a set molar ratio (e.g., 1:280:140) and subjected to multiple (e.g., 2, 3, 4, or more) temperature shifts (e.g., 10 minutes at room temperature, then 30° C. for 10 minutes), incubated (e.g., for 90 minutes). Detergent can be subsequently removed (e.g., by contact with a non-polar solid adsorbent). As one of many alternatives to the above procedure, one can use the method, or a variant thereof, as described in the Examples.

An integral membrane protein (including but not limited to a nucleic acid transporter protein as described herein) can be introduced into a apolipoprotein bound lipid bilayer. A variety of methods for introducing integral membrane proteins into apolipoprotein bound lipid bilayers have been described. See, e.g., U.S. Pat. Nos. 7,083,958 and 7,048,949; and Katzen et al., J. Proteome 7:3535-3542 (2008); Cappuccio et al., Mol. Cell. Proteomics 7:2246-2253 (2008); Banerjee et al., J. Mol. Biology 337(4): 1067-1081 (2008). Briefly, in some embodiments, the integral membrane protein is introduced into a apolipoprotein bound lipid bilayer by synthesizing the integral membrane (e.g., in a cell-free translation system) and contacting the synthesized integral membrane protein to the apolipoprotein bound lipid bilayer to incorporate the protein into the bilayer. Alternatively, the integral membrane protein can be added to the components of the apolipoprotein bound lipid bilayer during the bilayer formation, thereby incorporating the integral membrane protein into the bilayer as the bilayer is formed. One method of performing this latter option is provided in the Examples. Briefly, a tag (e.g., a poly-His tag) is included in either the scaffold protein or integral membrane protein, or both, and the proteins are then combined with an appropriate amount of lipid and detergent to form the apolipoprotein bound lipid bilayer. The tag can then be used to purify those bilayers containing the tagged protein.

### IV. Fusion of Lipid Bilayers to Cells

In some aspects of the present invention, an apolipoprotein bound lipid bilayer comprising an integral membrane protein is fused to the cell membrane of a cell, thereby allowing for introduction of the integral membrane protein into the cell membrane without expression (transcription or translation) of the protein by the cell itself.

The integral membrane of the cell can thus include a heterologous integral membrane protein. In some embodiments, the integral membrane protein will be one that is not encoded by the genome of the cell. For example, the integral membrane protein can be derived from a different species than the species of the cell or will be an artificial sequence.

In some embodiments, the cell will include a nucleic acid that encodes the introduced integral membrane protein. In some of these embodiments, the nucleic acid is not expressed (transcribed and/or translated). For example, the nucleic acid could be a pseudo gene. In some embodiments, the cell endogenously expresses the integral membrane protein (i.e., the exact amino acid sequence of the introduced integral membrane protein). For example, in some embodiments, a human cell will express human SID-1 or other nucleic acid transporter at a low level. In these cases, the cell membrane will include both endogenously-encoded integral membrane protein as well as exogenously introduced (i.e., via the apolipoprotein bound lipid bilayer) protein. In some embodiment, the amount of exogenous integral membrane protein is at least equal or greater than the amount of endogenous integral membrane protein having the same amino acid sequence.

Notably, where a cell has been fused with a apolipoprotein bound lipid bilayer of the invention, the cell may also include the scaffold apolipoprotein of the apolipoprotein bound lipid bilayer as well as the “payload” integral membrane protein. Thus, in some embodiments, cells fused with the apolipoprotein bound lipid bilayer are readily distinguished from native or naturally occurring cells by the presence of the scaffold apolipoprotein of the apolipoprotein bound lipid bilayer. According, in some embodiments, the invention provides for a cell comprising a scaffold apolipoprotein, as described herein, within the cell membrane of the cell, wherein the cell does not express the scaffold apolipoprotein.

Optionally, the apolipoprotein bound lipid bilayer and the target cell are fused without the addition of other agents that enhance fusion. Thus, in some embodiments, the apolipoprotein bound lipid bilayer and cell are fused in the presence of an isotonic buffer (e.g., PBS or other isotonic buffer). In some cases, additional Magnesium or calcium is included in the buffer. For example, in some embodiments, 0.1-10 mM, CaCl₂ and 0.1-10 mM MgCl₂ is used. Alternatively, in some embodiments, the apolipoprotein bound lipid bilayer and cell are fused in the presence of an agent that improves fusion. In some embodiments, for example, the quantity of apolipoprotein bound lipid bilayer that fuse to a cell is increased at least 10%, 50%, 100%, 200% or more compared to the absence of such an agent. Exemplary fusion enhancement agents include, but are not limited to polyethylene glycol (PEG) DMSO. For example, in some embodiments, up to 50% w/v PEG 300 or PEG 6000, micromolar to millimolar concentrations of Ca and/or Mg, and/or up to 5% DMSO are used to improve fusion.

It is believed that any type of cell without a cell wall can be used and fused with the apolipoprotein bound lipid bilayers of the invention. In some embodiments, the cells are animal cells, e.g., human cells or non-human cells (e.g., mammalian, mouse, rat, bovine, bird, primate, etc.).

Optionally, cells having an introduced integral membrane protein (and/or scaffold apolipoprotein) can be identified. For example, in some embodiments, after contacting the cell and apolipoprotein bound lipid bilayers (comprising an integral membrane protein) of the invention, an antibody or other agent that specifically binds the integral membrane protein or apolipoprotein can be used to detect cells having the target protein in their cell membrane. Optionally, cell sorting (e.g., FACS) can be employed to count or enrich for cells having the integral membrane or scaffold apolipoprotein.

Accordingly, invention provides for cells that are products of fusion with the apolipoprotein bound lipid bilayers of the invention. Such cells will comprise the exogenous integral membrane protein in the cell membrane as introduced via the apolipoprotein bound lipid bilayers of the invention products. These cells will therefore have an integral membrane protein present in their cell membrane without comprising a nucleic acid (e.g., RNA, genomic DNA, viral DNA, plasmid DNA, etc.) encoding the protein. Alternative, the protein will be encoded by the cell, but the membrane will comprise copies of the integral membrane polypeptide that were not translated in the cell. For example, the resulting cell will comprise more copies of the integral membrane polypeptide than would occur to a cell under similar conditions that was not fused with the NABBs as described herein.

V. Transformation with Nucleic Acids

In embodiments in which a nucleic acid integral membrane protein is introduced into the cell membrane of a cell, the invention further provides for contacting such cells with a nucleic acid thereby allowing for introduction of the nucleic acid into the cell. The length, composition, and concentration of the nucleic acids contacted to the cells comprising the nucleic acid transporters, as described herein, will depend on the specific nucleic acid transporter, e.g., whether it transports all nucleic acids, nucleic acids of a specific size, double or single stranded nucleic acids, RNA, DNA, and/or mimetics thereof, etc.

Where the nucleic acid transporter is capable of transporting RNA, the cell can be contacted with an RNA molecule or a mimetic thereof. RNA nucleic acids can be either double stranded, single-stranded or both. Functionally, in some embodiments, the RNA mediate RNA interference in the cell. RNA interference (RNAi) is normally triggered by double stranded RNA (dsRNA) or endogenous microRNA precursors (pre-miRNAs). In some embodiments, the RNA are siRNAs, hnRNAs, microRNAs or other RNAs.

MicroRNAs (miRNAs) are endogenously encoded ˜22-nt-long RNAs that are generally expressed in a highly tissue- or developmental-stage-specific fashion and that post-transcriptionally regulate target genes. More than 200 distinct miRNAs having been identified in plants and animals, these small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). miRNAs can be expressed in a highly tissue-specific or developmentally regulated manner and this regulation likely plays a role in eukaryotic development and differentiation.

miRNAs are first transcribed as part of a long, largely single-stranded primary transcript (Lee et al., EMBO J. 21: 4663-4670, 2002). This primary miRNA transcript is generally, and possibly invariably, synthesized by RNA polymerase II (pol II) and therefore is normally polyadenylated and may be spliced. It contains an .about.80-nt hairpin structure that encodes the mature .about.22-nt miRNA as part of one arm of the stem. In animal cells, this primary transcript is cleaved by a nuclear RNaseIII-type enzyme called Drosha (Lee et al., Nature 425: 415-419, 2003) to liberate a hairpin miRNA precursor, or pre-miRNA, of .about.65 nt, which is then exported to the cytoplasm by exportin-5 and the GTP-bound form of the Ran cofactor (Yi et al., Genes Dev. 17: 3011-3016, 2003). Once in the cytoplasm, the pre-miRNA is further processed by Dicer, another RNaseIII enzyme, to produce a duplex of .about.22 bp that is structurally identical to an siRNA duplex (Hutvagner et al., Science 293: 834-838, 2001). The binding of protein components of the RNA-induced silencing complex (RISC), or RISC cofactors, to the duplex results in incorporation of the mature, single-stranded miRNA into a RISC or RISC-like protein complex, whereas the other strand of the duplex is degraded (Bartel, Cell 116: 281-297, 2004).

An miRNA can be completely complementary or can have a region of noncomplementarity with a target nucleic acid, consequently resulting in a “bulge” at the region of non-complementarity. The region of noncomplementarity (the bulge) can be flanked by regions of sufficient complementarity, preferably complete complementarity to allow duplex formation. In some embodiments, the regions of complementarity are at least 8 to 10 nucleotides long (e.g., 8, 9, or 10 nucleotides long). A miRNA can inhibit gene expression by repressing translation, such as when the microRNA is not completely complementary to the target nucleic acid, or by causing target RNA degradation, which is believed to occur only when the miRNA binds its target with perfect complementarity. The invention also can include double-stranded precursors of miRNAs that may or may not form a bulge when bound to their targets.

Given a sense strand sequence (e.g., the sequence of a sense strand of a cDNA molecule), an miRNA can be designed according to the rules of Watson and Crick base pairing. The miRNA can be complementary to a portion of an RNA, e.g., a miRNA, a pre-miRNA, a pre-mRNA or an mRNA. For example, the miRNA can be complementary to the coding region or noncoding region of an mRNA or pre-mRNA, e.g., the region surrounding the translation start site of a pre-mRNA or mRNA, such as the 5′ UTR. An miRNA oligonucleotide can be, for example, from about 12 to 30 nucleotides in length, preferably about 15 to 28 nucleotides in length (e.g., 16, 17, 18, 19, 20, 21, 22, 23, 24, or 25 nucleotides in length).

In some embodiments, a cell comprising an exogenous nucleic acid transporter as described herein is contacted with a double stranded RNA under conditions allowing for entry of the dsRNA into the cell via the transporter. The term “double-stranded RNA” or “dsRNA”, as used herein, refers to a complex of ribonucleic acid molecules, having a duplex structure comprising two anti-parallel, and at least substantially complementary, nucleic acid strands. The two strands forming the duplex structure may be different portions of one larger RNA molecule, or they may be separate RNA molecules. Where separate RNA molecules, such dsRNA are often referred to in the literature as siRNA (“short interfering RNA”). Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”, “short hairpin RNA” or “shRNA”. Where the two strands are connected covalently by means other than an uninterrupted chain of nucleotides between the 3′-end of one strand and the 5′ end of the respective other strand forming the duplex structure, the connecting structure is referred to as a “linker”.

In some embodiments, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). The dsRNA can be synthesized by standard methods known in the art as further discussed below, e.g., by use of an automated DNA synthesizer, such as are commercially available from, for example, Biosearch, Applied Biosystems, Inc.

In some embodiments, the nucleotides of an RNA or DNA molecule contacted to the cell as described herein comprises at one or both strands, a modification to prevent or inhibit the degradation activities of cellular enzymes, such as, for example, without limitation, certain nucleases. Techniques for inhibiting the degradation activity of cellular enzymes against nucleic acids are known in the art including, but not limited to, 2′-amino modifications, 2′-amino sugar modifications, 2′-F sugar modifications, 2′-F modifications, 2′-alkyl sugar modifications, 2′-O-alkoxyalkyl modifications like 2′-O-methoxyethyl, uncharged and charged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, generally by a 2′-F or a 2′-O-methyl group. Also, at least one nucleotide may be modified to form a locked nucleotide. Such locked nucleotide contains a methylene bridge that connects the 2′-oxygen of ribose with the 4′-carbon of ribose. Oligonucleotides containing the locked nucleotide are described in Koshkin, A. A., et al., Tetrahedron (1998), 54: 3607-3630) and Obika, S. et al., Tetrahedron Lett. (1998), 39: 5401-5404). Introduction of a locked nucleotide into an oligonucleotide improves the affinity for complementary sequences and increases the melting temperature by several degrees (Braasch, D. A. and D. R. Corey, Chem. Biol. (2001), 8:1-7).

### EXAMPLES

The following examples are offered to illustrate, but not to limit the claimed invention.

### Example I

### Incorporating the Protein SID-1 into NABBs and its Use as a Tool for RNAi Introduction Expression of SID-1

The gene for the C. elegans protein SID-1 or one of its homologs or orthologs is cloned into a standard plasmid (such as pBR322 or related vector) under appropriate promoter control (for example lacZ) and tagged for downstream isolation (for example, His tag). The SID-1 plasmid is transformed into E. coli.

E. coli colonies containing the SID-1 plasmid are induced to express the protein in a fashion consistent with the promoter type. SID-1 is isolated from E. coli, e.g., via detergent solublization (for example, using CHAPS or Sodium Deoxycholate) and purified from other bacterial proteins using the biological tag. Purified SID-1 solublized in detergent is combined with phospholipids in the procedure outlined below to create NABBs that contain SID-1.

### Example 2

### Incorporating SID-1 into NABBs

A lipid stock solution is made as follows:
a. POPC stock: [POPC]f=100 mg/ml in 10% NaCHolate, 20 mM Tris HCl, pH 8/150 mM NaCl buffer.
b. NBD stock: [NBD]f=1 mg/ml in 1.5% NaCholate, 20 mM Tris HCl, pH 8/150 mM NaCl buffer.

Lipid solutions are dipped into LN₂ bath until it freezes (˜30-60 sec). The tube is left at room temperature for ˜2 minutes. The tube is plunged (lipid is still be frozen) into a room temperature water bath and swirled until solution thaws. The above process is repeated 2-3 times the solutions are clear. The detergent & lipid stocks can be stored at −20° C., buffer at 4° C., over night.

### Cell Lysate Preparation:

A ZAP1 stock is thawed and spun for 2 min at greater of equal to 16000×g to pellet any debris. The final concentrations of lipids and ZAP1 for NABB formation are as follows:

1. 0.5% POPC

2. 86 μM ZAP1

3. 0.005% NBD-DOPE

Ideally, the solution has a Lipid:ZAP1 ratio of 75:1 and a POPC:NPD ratio of 100:1.

### NABB Preparation:

100 μL NABB Reaction is prepared as follows:

5 μL 10% POPC

29.5 μL 291 uM ZAP1

5 μL 0.1% NBD DOPE

60.5 μL of SID-1

The reaction solution is vortexed 3×30 seconds and left at room temperature for 15-30 minutes. The solution is then centrifuged at 5000×g for 5 minutes. The supernatant is taken and applied to pre-equilibrated Extracti Gel D column (Pierce Scientific). The sample is allowed to flow all the way into the column. A volume of Tris/NaCl buffer (no detergent) is added that is 2× the size of the sample to the column. (i.e. 100 μL NABB r×n, add 200 buffer). A 200 μL flow through fraction is collected. This wash is repeated five times.

SID-1 containing NABBs are eluted in the void volume. NABBs are monitored by monitoring protein absorbance or lipid fluorescence.

### SID-1 Containing NABB Purification Via ZAP-1 His-Tag

Take 1 ml of IMAC bead stock, spin at 5000×g for 2 minutes, and remove supernatant. An equal volume of Tris/NaCl buffer is added and incubated at room temperature with nutation for ˜10 minutes. The above steps are then repeated one more time. The final supernatant is removed and NABB eluate is added to IMAC beads and incubated at 4° C. for 60 minutes with nutation. The beads are then centrifuged and pelleted (6000×g, 5 min). The resulting supernatant is removed and washed 3×250 μL, spinning as above for each wash. The bound NABBs are eluted using Tris buffer with 10 mM EDTA in 2×200 μl volumes with nutation for 15 minutes each time.

### Example 3

### Using SID-1 Containing NABBs as a Transfection Reagent

Diafiltration is used to suspend the SID-1-containing NABBs in a solution (for example, PBS-based solutions containing micromolar to millimolar concentrations of calcium and/or magnesium (e.g., 0.9 mM CaCl₂ and 0.5 mM MgCl₂), polyethyleneglycol, DMSO, pyrene butyrate or similar compounds) to encourage fusion of the NABBs with the cell membrane. Media is removed from cells and SID-1 NABBs are added and incubated for 10-15 minutes. The media is then replaced and a solution containing an siRNA or dsRNA of interest is added and cells are returned to standard growth conditions (37° C./5% CO₂ incubator). RNAi activity is analyzed after approximately 6 hours or more.

### Example 4

Conditions for NABBs and NaBBs containing an integral membrane protein (Boivne rhodopsin) to integrate into the plasma membrane of a mammalian cell were determined as follows.

Initially, NABBs (see, e.g., U.S. Pat. No. 7,083,958) without an integral membrane protein, but with an detectable label, were used to demonstrate fusion with cell membranes. A method for labeling the NABB particles using the fluorescent lipophilic dye DiO was developed. The protocol involved 30 minute incubation of NABBs with the dye on ice followed by centrifugal diafiltration to remove excess dye. DiO-labeled NABBs were added to cells under a number of conditions and at different concentrations in order to determine the optimal conditions for NABB integration. Buffers tested were: 1-15% DMSO, 5-15% PEG 300, 5-15% PEG 6000, PBS, and PBS containing 0.9 mM CaCl₂ and 0.5 mM MgCl₂. Successful NABB uptake was observed in cells with DMSO, PBS, and PBS with CaCl₂ and MgCl₂. Of the buffers tested, the optimal solution for uptake was PBS with CaCl₂ and MgCl₂.

Time course experiments showed that NABBs can be taken into the cells in as little as 2 minutes under optimal conditions and that they can stay in the cell for at least 8 hours. Optimal uptake was achieved when NABBs were incubated with cells for 15 minutes. Experiments demonstrated that NABB uptake occurs between 4° C.-37° C. in a temperature dependent fashion (i.e., the colder the cells, the slower the rate of uptake). NABBs were taken up by multiple cell types: HeLa (human cervical cells), CHO (Chinese hamster ovary), and COS (African green monkey cells), demonstrating that this method has broad applicability among mammalian cells. Confocal microscopy demonstrated that the labeled NABBs can be found on the cell surface.

In addition, NABBs comprising rhodopsin were tested for their ability to deliver an integral membrane protein to the cell membrane of cells. Optimal conditions as determined by the NABB-only experiments were used (PBS+Ca, +Mg, 15 minutes for uptake). The presence of rhodopsin and its location in the cell was tracked using an antibody that recognizes the extracellular domain of rhodopsin. Confocal microscopy revealed that the integral membrane protein was delivered by the NABBs and localized primarily to the cell surface (plasma membrane).

The DiO-labeled lipid from the NABBs colocalized with the rhodopsin at early time points (less than 30 minutes), demonstrating that delivery of the integral membrane protein was via the NABB particles. Rhodopsin uptake was demonstrated at concentrations between 1-10 μM. The optimum concentration was determined by the nature of the experiment and the type of functionality one was monitoring for the membrane protein of choice. Therefore the concentration range for the NABB-protein particles could be quite broad, e.g., 1 nM-100 μM. Expression of rhodopsin on the cell surface was at its peak between 30-120 minutes post-addition of the NABB/rhodopsin particles. Most of the expression (>80%) was gone from the cell surface by 330 minutes. Cells exposed to free rhodopsin (i.e. that was not contained in NABBs) did not incorporate rhodopsin into the cell membrane.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated cell comprising a cell membrane, the membrane comprising an integral membrane polypeptide, wherein the integral membrane polypeptide is heterologous to the cell and the cell does not comprise a nucleic acid encoding the polypeptide, or wherein the membrane comprises copies of the integral membrane polypeptide that were not translated in the cell.

2. The isolated cell of claim 1, wherein the cell does not comprise a nucleic acid encoding the polypeptide.

3. The isolated cell of claim 1, wherein the membrane comprises copies of the integral membrane polypeptide that were not translated in the cell.

4. The isolated cell of claim 1, wherein the integral membrane polypeptide is a nucleic acid transporter polypeptide.

5. The isolated cell of claim 4, wherein the nucleic acid transporter polypeptide is an RNA transporter.

6. The isolated cell of claim 4, wherein the nucleic acid transporter polypeptide comprises an amino acid sequence at least 80% identical to any of SEQ ID NOs:1-21.

7. The isolated cell of claim 4, wherein the nucleic acid transporter polypeptide comprises any of SEQ ID NOs:1-21.

8. The isolated cell of claim 1, wherein the cell is mammalian cell.

9. A method of making the cell of claim 1, the method comprising
contacting a cell having a cell membrane with an apolipoprotein bound lipid bilayer comprising the integral membrane polypeptide under conditions to allow for fusion of the lipid bilayer to the cell membrane, thereby introducing the polypeptide into the cell membrane.

10. The method of claim 9, wherein the integral membrane protein is a nucleic acid transporter polypeptide.

11. The method of claim 10, wherein the nucleic acid transporter polypeptide is an RNA transporter.

12. The method of claim 10, wherein the nucleic acid transporter polypeptide comprises an amino acid sequence at least 80% identical to any of SEQ ID NOs:1-21.

13. The method of claim 10, wherein the nucleic acid transporter polypeptide comprises any of SEQ ID NOs:1-21.

14. The method of claim 9, wherein the contacting step comprising contacting the cell with an agent that enhances fusion in the presence of the lipid bilayer.

15. The method of claim 14, wherein the agent is selected from the group consisting of polyethylene glycol (PEG), dimethyl sulfoxide (DMSO), pyrene butyrate, and phosphate buffered saline either with or without supplementary divalent and/or monovalent salts.

16. A method of introducing an exogenous nucleic acid into a cell, the method comprising
contacting the cell of claim 4 with an exogenous nucleic acid, thereby introducing the exogenous nucleic acid into the cell.

17. The method of claim 16, wherein the polypeptide is an RNA transporter and the exogenous nucleic acid comprises RNA.

18. The method of claim 16, wherein the polypeptide is an RNA transporter and the exogenous nucleic acid comprises double stranded RNA.

19. The method of claim 16, wherein the polypeptide is an RNA transporter and the exogenous nucleic acid is an siRNA.

20. An apolipoprotein bound lipid bilayer comprising a nucleic acid transporter polypeptide.

21. The apolipoprotein bound lipid bilayer of claim 20, wherein the nucleic acid transporter polypeptide is an RNA transporter.

22. The apolipoprotein bound lipid bilayer of claim 20, wherein the nucleic acid transporter polypeptide comprises an amino acid sequence at least 80% identical to any of SEQ ID NOs:1-21.

23. The apolipoprotein bound lipid bilayer of claim 20, wherein the nucleic acid transporter polypeptide comprises any of SEQ ID NOs:1-21.
